(19) United States
(12) Patent Application Publication
(10) Pub. No.: US 2025/0281102 A1
(43) Pub. Date: Sep. 11, 2025

(54) SYSTEM AND METHOD FOR CLINICAL DISORDER ASSESSMENT

(71) Applicant: The General Hospital Corporation, Boston, MA (US)

(72) Inventor: Anoopum S. Gupta, Newton, MA (US)

(73) Assignee: The General Hospital Corporation, Boston, MA (US)

(21) Appl. No.: 18/719,106

(22) PCT Filed: Dec. 12, 2022
(86) PCT No.: PCT/US2022/081374

§ 371 Date: Jun. 12, 2024

Related U.S. Application Data
(60) Provisional application No. 63/288,619, filed on Dec. 12, 2021.

(51) Int. Cl. A61B 5/00; A61B 5/11; G16H 15/00; G16H 50/20

(52) U.S. Cl. CPC A61B 5/4082; A61B 5/1128; A61B 5/6824; A61B 5/6829; A61B 5/7264; A61B 5/7271; G16H 15/00; G16H 50/20; A61B 2560/0462; A61B 2562/0219

(57) ABSTRACT

A system and method for clinical disorder assessment are disclosed. The method and the medical assessment system using the method include: obtaining sensor data indicative of movement of a user; generating a movement dataset by reducing dimensions of the sensor data; generating a plurality of submovement datasets based on the movement dataset; extracting a movement feature from a first subset of the plurality of submovement datasets; analyzing the movement feature from the first subset of the plurality of submovement datasets to a reference to determine a potential clinical disorder of the user; and generating a report that includes an indication and severity of the potential clinical disorder of the user. Other aspects, embodiments, and features are also claimed and described.

### CROSS-REFERENCE TO RELATED APPLICATIONS

This application is based on, claims priority to, and incorporates herein by reference in their entirety U.S. Provisional Application Ser. No. 63/288,619, filed Dec. 12, 2021.

### BACKGROUND

The present disclosure relates generally to systems and methods for assessing clinical disorders. More particularly, the present disclosure provides systems and methods for neurological disease assessment based on movement measures of patients.

The development of new therapeutic modalities is accelerating for clinical disorders including neurological disorders with a large unmet medical need. However, currently used tools for determining the efficacy of therapies remain subjective, imprecise, and insensitive. In addition, the clinician-performed therapies capture the state of the individual at a snapshot in time. Thus, when measurements are collected infrequently, as is the case for in-person assessments, these tools cannot account for day-to-day and moment-to-moment variability in the disease state, and have limited ability to account for variability in behavioral task performance and measurement error. Furthermore, it can be unclear whether the measured disease characteristics reflect aspects of behavioral change that are meaningful to patients. Therefore, it would be desirable to have a system and method for objectively and more precisely assessing clinical disorders.

### SUMMARY

The present disclosure overcomes the aforementioned drawbacks by providing systems and methods for clinical disorder assessment based on interpretable submovement features that have specific relevance to the control of movement.

In accordance with one aspect of the present disclosure, a medical assessment system is provided that includes an input configured to receive sensor data indicative of movement of a subject, a memory, and a processor coupled to the memory. The processor is configured to: receive the sensor data indicative of movement of the subject; generate a plurality of submovement datasets using the sensor data; extract a movement feature from a first subset of the plurality of submovement datasets; analyze the movement feature from the first subset of the plurality of submovement datasets to determine a potential clinical disorder of the user; and generate a report that indicates the potential clinical disorder of the user.

In accordance with another aspect of the present disclosure, a method is provided for clinical disorder assessment. The method includes: receiving sensor data indicative of movement of the subject; generating a plurality of submovement datasets using the sensor data; extracting a movement feature from a first subset of the plurality of submovement datasets; analyze the movement feature from the first subset of the plurality of submovement datasets to determine a potential clinical disorder of the user; and generate a report that indicates the potential clinical disorder of the user.

The foregoing and other advantages of the inventions will appear in the detailed description that follows. In the description, reference is made to the accompanying drawings.

### BRIEF DESCRIPTION OF THE DRAWINGS

Implementations of the invention will become more apparent from the detailed description set forth below when taken in conjunction with the drawings, in which like elements bear like reference numerals.

FIG. 1 is diagram of one, non-limiting example of a system in accordance with the present disclosure.

FIG. 2 is a flow chart setting forth some, non-limiting example steps of a process in accordance with the present disclosure that may utilize a system such as described with respect to FIG. 1.

FIG. 3A is graphs illustrating one night and day of accelerometer data from sensors.

FIG. 3B is a graph showing partial daytime accelerometer data of the data in FIG. 3A.

FIG. 4 is graphs of triaxial velocity data velocity converted from an activity bout identified within the accelerometer data of FIG. 3B in accordance with the present disclosure.

FIG. 5 is graphs of the velocity data of FIG. 4 on a two-dimensional plane in accordance with the present disclosure.

FIGS. 6 and 7 is graphs of multiple submovement datasets in accordance with the present disclosure.

FIG. 8 is graphs representing low-frequency and high-frequency characteristics of the velocity-time curve of the multiple submovement datasets in accordance with the present disclosure.

FIGS. 9A-9F shows properties of a single ankle submovement feature in accordance with the present disclosure.

FIGS. 10A-10F shows properties of a Hevelius composite model in accordance with the present disclosure.

FIG. 11 shows the relationship between each wrist sensor feature subset and key clinical comparisons in accordance with the present disclosure.

FIGS. 12A-12X show normalized histograms of long duration submovement properties for individuals with ataxia-telangiectasia versus controls in accordance with the present disclosure.

FIGS. 13A-13D show the relationships between submovement peak velocity and submovement distance (FIGS. 13A and 13B) and submovement duration versus distance (FIGS. 13C and 13D), separately for controls and individuals with ataxia-telangiectasia for long duration submovements in accordance with the present disclosure.

### DETAILED DESCRIPTION

The present disclosure recognizes that properties or characteristics of motor primitives called “submovements” can be used to assess the potential for a patient suffering from a clinical disorder. More particularly, data for analysis can be derived from the continuous wearable sensors or other sources, such as video, that is significantly correlated with clinical disorder severity. With this in mind, the present disclosure provides systems and methods for assessing motor function in clinical disorders as well as in healthy populations during childhood development, the process of aging, and in response to interventions such as diet and exercise. More particularly, the present disclosure provides systems and methods for clinical disorder assessment based on submovement features extracted from one or more wearable devices (e.g., smart wrist band, smart ankle band, etc.) or other sensors including video sensors. Thus, the present disclosure recognizes that the example method and/or the medical assessment system can determine a potential clinical disorder (e.g., a neurodegenerative disorder, a movement disorder, abnormal childhood development, or any suitable neurological disease or disorder) and/or the severity of the potential clinical disorder.

### Example Medical Assessment System

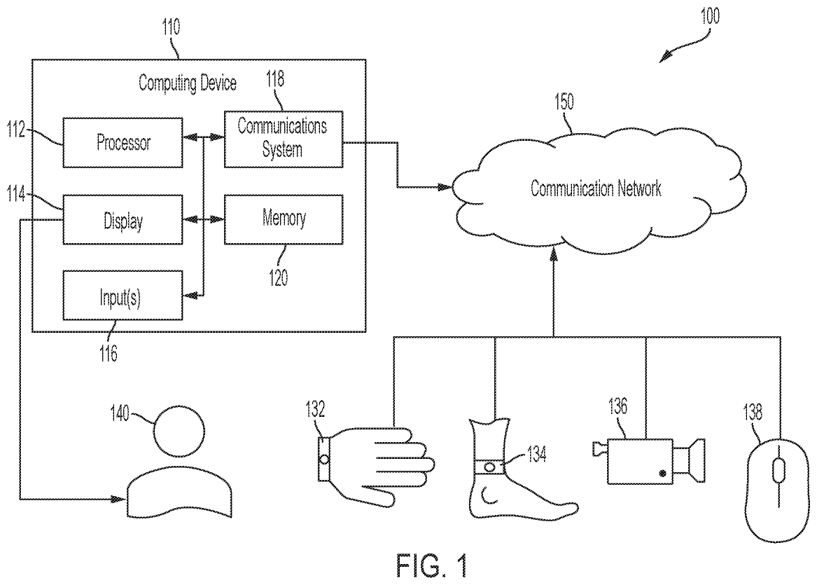

FIG. 1 shows a block diagram illustrating a medical assessment system for clinical disorder assessment according to some embodiments. As shown in FIG. 1, computing device 110 can include an input 111 to receive sensor data from data sources (e.g., one or more wearable devices (smart wrist band 132, smart ankle band 134, a virtual reality headset, etc.), camera 136, a video game controller, a mobile device, and/or any other suitable device), generate movement features based on the submovement datasets to determine a potential clinical disorder, and provide a report including an indication of the potential clinical disorder to a user, subject, patient, or potential patient 140. As will be explained, the report can include measures of motor performance and/or longitudinal data relative to test or re-test consistency or performance changes.

In some examples, computing device 110 can include processor 112 can include processor 112. In some embodiments, the processor 112 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), etc.

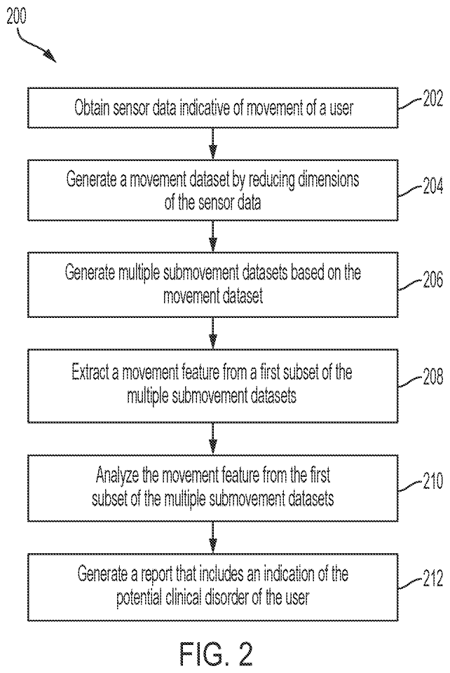

In further examples, computing device 110 can further include a memory 120. The memory 120 can include any suitable storage device or devices that can be used to store suitable data (e.g., sensor data, submovement datasets, movement feature(s), regression model(s) etc.) and instructions that can be used, for example, by the processor 112 to obtain sensor data indicative of movement of a user, generate a movement dataset by reducing dimensions of the sensor data, generate a plurality of submovement datasets based on the movement dataset, extract a movement feature from a first subset of the plurality of submovement datasets, compare the movement feature from the first subset of the plurality of submovement datasets to a reference to determine a potential clinical disorder of the user, generate a report. The report can include an indication of the potential clinical disorder of the user, project the sensor data on a two-dimensional plane, divide the movement dataset into the plurality of submovement datasets based on one or more zero crossings of the movement dataset, group the plurality of submovement datasets into a plurality of subsets based on a duration and a direction of the movement of the user in the plurality of submovement datasets, obtain a regression model trained with the reference, provide the movement feature to the regression model, and/or generate an output of the regression model to determine the potential clinical disorder of the user. The memory 120 can include any suitable volatile memory, non-volatile memory, storage, or any suitable combination thereof. For example, memory 120 can include random access memory (RAM), read-only memory (ROM), electronically-erasable programmable read-only memory (EEPROM), one or more flash drives, one or more hard disks, one or more solid state drives, one or more optical drives, etc. In some embodiments, the memory 120 can have encoded thereon a computer program for generating a virtual reality environment, calibrating the virtual reality environment to a user, displaying components of the therapeutic game in the virtual reality environment, etc. For example, in such embodiments, the processor 112 can execute at least a portion of the computer program to perform one or more data processing tasks described herein transmit/receive information via the communications system(s) 118, etc. As another example, the processor 112 can execute at least a portion of process 200 described below in connection with FIG. 2.

In further examples, computing device 110 can further include communications system 118. Communications system 118 can include any suitable hardware, firmware, and/or software for communicating information over communication network 140 and/or any other suitable communication networks. For example, communications system 118 can include one or more transceivers, one or more communication chips and/or chip sets, etc. In a more particular example, communications system 118 can include hardware, firmware and/or software that can be used to establish a Wi-Fi connection, a Bluetooth connection, a cellular connection, an Ethernet connection, etc.

In further examples, computing device 110 can receive or transmit information from or to data source(s) (e.g., a smart wrist band 132, a smart ankle band 134, a camera 136, a virtual reality headset, a game controller, a mobile device, or any other suitable movement sensing device) and/or any other suitable system over a communication network 150. In some examples, the communication network 150 can be any suitable communication network or combination of communication networks. For example, the communication network 150 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In some embodiments, communication network 150 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

In further examples, computing device 110 can further include a display 114 and/or one or more inputs 116. In some embodiments, the display 114 can include any suitable display devices, such as a computer monitor, a touchscreen, a television, an infotainment screen, etc. to display the report or any suitable clinical disorder assessment to the user 140. In further embodiments, the input(s) 116 can include any suitable input devices and/or sensors that can be used to receive user input, such as a keyboard, a mouse, a touchscreen, a microphone, etc. In further embodiments, the input(s) 116 can include data source(s) (e.g., a smart wrist band 132, a smart ankle band 134, a camera 136, a mouse 138, etc.) and directly receive the sensor data. However, due to limited system resources (e.g., memory, processing, bandwidth, energy, etc.), the sensor node 110a, 110n might not include a display 114 or one or more inputs 116.

### Example Process

FIG. 2 is a flow diagram illustrating an example process for clinical disorder assessment according to some embodiments. As described below, a particular implementation can omit some or all illustrated features/steps, may be implemented in some embodiments in a different order, and may not require some illustrated features to implement all embodiments. In some examples, a computing device 110 in connection with FIG. 1 can be used to perform the example process 200 (e.g., by processor 112 executing instructions stored in memory 120 for performing process 200). However, it should be appreciated that any suitable apparatus or means for carrying out the operations or features described below may perform the process 200.

In some examples, the process 200 assesses a clinical disorder. The clinical disorder can include a neurodegenerative disease or disorder (e.g., Alzheimer's disease, Parkinson's disease, Huntington's disease, Multiple sclerosis, Amyotrophic lateral sclerosis, Batten disease, Creutzfeldt-Jakob disease, etc.), a movement disorder (e.g., ataxia, dystonia, essential tremor, Huntington's disease, multiple system atrophy, myoclonus, Parkinson's disease, progressive supranuclear palsy, Rett syndrome, secondary Parkinsonism, spasticity, tardive dyskinesia, Tourette syndrome, Wilson's disease, etc.), or any suitable neurological disease (e.g., stroke, traumatic brain injury, concussion, developmental delay, premature aging, etc.) or non-neurological disorder that restricts or changes the quality of movement (e.g., arthritis, chronic heart failure, chronic obstructive pulmonary disease, etc.). Notably, the sensor data may be acquired differently based on the clinical disorder(s) being analyzed. For example, sampling frequency or sensitivity may be adjusted. Furthermore, as will be discussed, the analysis of the sensor data may be adjusted to assess each or selected clinical disorders.

At process block 202, the process 200 obtains sensor data indicative of movement of a user. For example, the sensor data can include velocity data. In some examples, the velocity data can be converted from acceleration data or from position data. In further examples, the acceleration data can be received from one or more wearable sensor devices on at least one of a wrist or an ankle of the user. For example, the user can use one wearable sensor device (e.g., a smart wrist band) for the dominant wrist of the user and another wearable sensor device (e.g., a smart ankle band) for the dominant ankle of the user. The wearable sensor device can include an accelerometer, which produces the acceleration data. In some examples, the acceleration data can be triaxial acceleration data in three orthogonal directions, and the velocity data can be triaxial velocity data. However, the acceleration data can be single- or other multiple-axis acceleration data. In addition, it should be appreciated that the acceleration data can be received from any other suitable means. For example, the process 200 can receive video data and obtain the acceleration data based on the video data or multiple images (e.g., by extracting and tracking joints of the individual in the video data or multiple images and generating the acceleration data based on the tracked joint movements in time-series). Further, it should be understood that the sensor data is not limited to the velocity data in velocity-time dimensions. For example, the sensor data can be data in acceleration-time dimensions, location-time dimensions, or any data in suitable dimensions. In further examples, the sensor data can include videos, a series of pictures of the user, acceleration data, or any other suitable data.

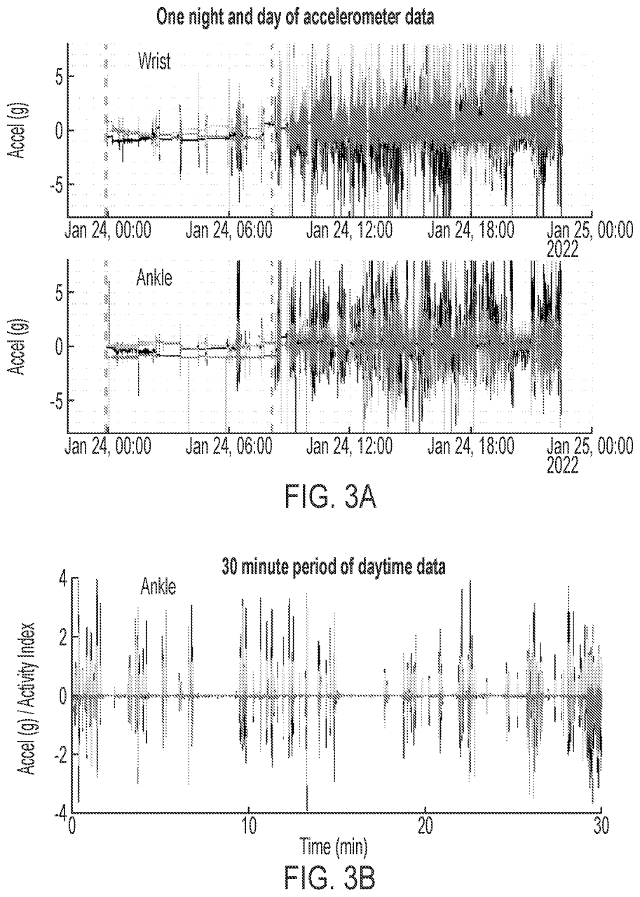

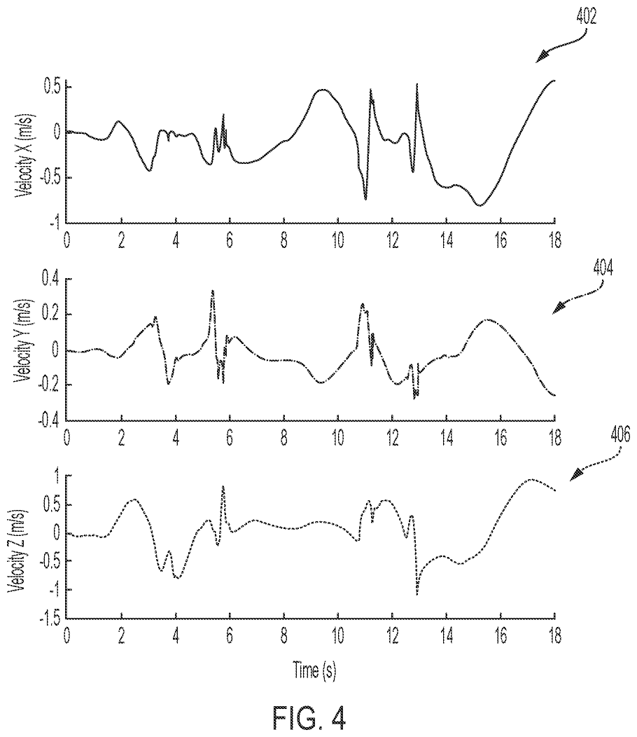

Referring to FIG. 3A, the sensor data includes triaxial acceleration data during one night and day for each of a wrist and an ankle of the user. Thus, the triaxial acceleration data can include an x-axis acceleration dataset, a y-axis acceleration dataset, and a z-axis acceleration dataset for each of the wrist and the ankle of the user (i.e., three acceleration datasets for the wrist of the user and three other acceleration datasets for the ankle of the user). Referring to FIG. 3B, the sensor data includes triaxial acceleration data for 30 minutes of daytime or any other suitable time period. Referring to FIG. 4, the sensor data can include triaxial velocity data (i.e., an x-axis velocity dataset 402, a y-axis velocity dataset 404, and a z-axis velocity dataset 406). In some examples, the triaxial velocity data in FIG. 4 is converted from the triaxial acceleration data shown in FIG. 3A or 3B. In some examples, the sensor data can be normalized (e.g., 0 to 2 range, −1 to 1 range, or any other suitable range).

In some examples, the sensor data can be continuous data for a predetermined period of time. For example, the sensor data can include data for one or more wearable sensors (e.g., for wrist, ankle, and/or any other suitable body location of the user) for one night and day as shown in FIG. 3A, daytime, one or more hours, one or more minutes (e.g., 30 minutes as shown in FIG. 3B), one or more seconds (e.g., 18 seconds as shown in FIG. 4), or any other suitable time period. In further examples, the sensor data can include a single activity bout for a corresponding period of time. For example, the single activity bout can be defined as acceleration or velocity values between two adjacent times with 0 acceleration values or 0 velocity values in all directions. Thus, the period of time for the sensor data can be adaptably defined based on the movement or activity of the user. In some examples, the processing blocks 204-212 below can be applicable to the sensor data for the time period. In other examples, the processing blocks 204-210 below can be applicable to multiple sets of the sensor data to generate a report at the processing block 212. The report can indicate a potential clinical condition, measures of motor performance, and/or test/retest consistency, as well as other information, such as will be described.

Referring again to FIG. 2, at process block 204, the process 200 generates a movement dataset by reducing dimensions of the sensor data. In some examples, to reduce the dimensions of the sensor data, the process 200 can project the sensor data on a two-dimensional plane. In further examples, the process 200 can project movement of the sensor data onto a two-dimensional plane for each principal axes of the planar projection. In even further examples, the process 200 can use principal component analysis (PCA) to reduce dimensions of the sensor data. For example, the movement dataset can include a first principal component dataset (e.g., PC1 dataset). in a primary direction. The primary direction has the maximum movement variation of the sensor data. In further examples, the movement dataset can further include a second principal component dataset (e.g., PC2 dataset) in a secondary direction. The secondary direction can be orthogonal to the primary direction. In further examples, the process 200 can normalize the movement dataset to have values in the movement dataset on different scales (e.g., into the range between −1 and 1 or any other suitable range).

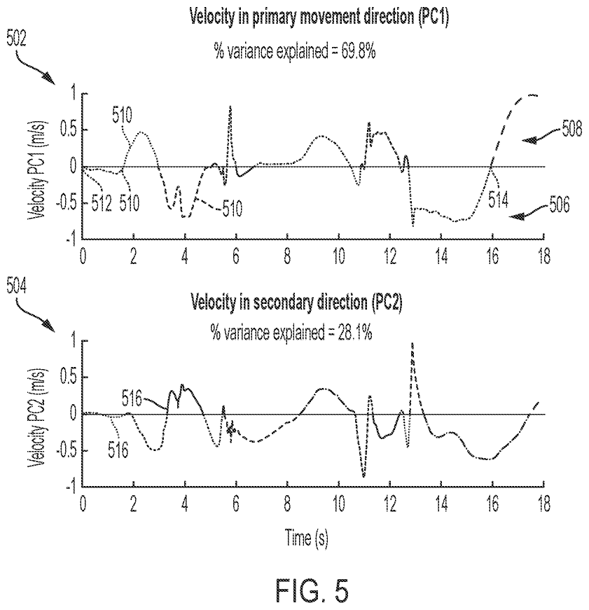

Referring to FIG. 5, example movement data (e.g., the first principal component dataset 502 and the second principal component dataset 504) is shown on a two-dimensional plane (e.g., the velocity-time space). In the examples in FIG. 5, the first principal component dataset 502 is the sensor data (e.g., the triaxial velocity data in FIG. 4) projected onto the velocity-time space with the primary direction of the movement in the sensor data. In the examples, the primary direction is the direction where the largest variance (69.8% variance) in the velocity-time curve of the sensor data (e.g., the triaxial velocity data) occurs. In the examples, the second principal component dataset 504 is the sensor data (e.g., the triaxial velocity data in FIG. 4) projected onto the velocity-time space with the secondary direction of the movement in the sensor data. In the examples, the secondary direction is orthogonal to the primary direction. The variance in the velocity-time curve of the sensor data in FIG. 5 is 28.1%. It should be appreciated that the process 200 can use another suitable technique (e.g. nonlinear dimensionality reduction approaches, Isomap, locally-linear embedding (LLE), neural network autoencoders, etc.) to reduce dimensions of the sensor data. In other examples, process block 204 can be optional. Thus, process 200 can proceed with process block 206 without reducing dimensions of the sensor data.

Referring again to FIG. 2, at process block 206, the process 200 generates multiple submovement datasets based on the movement dataset. For example, the process 200 can divide the movement dataset into the multiple submovement datasets based on one or more zero crossings of the movement dataset. A first submovement dataset can be a dataset between two abutting zero velocity crossings in the movement dataset. In some examples, the beginning and/or the end of a submovement dataset can be identified by a time where the values of the movement dataset changes (e.g., from a positive value to a negative value or from a negative value to a positive value). Then the submovement dataset includes homogeneous values (e.g., 0 and/or positive values of the movement dataset or 0 and/or negative values of the movement dataset). In further examples, the beginning or the end of some submovement dataset can be defined by the beginning or the end of the movement dataset rather than changes of the movement dataset. In further examples, the beginning or end of a submovement can be inferred based on computational models of submovements, allowing for temporally overlapping submovements to be detected. In even further examples, a submovement dataset can be normalized to have values in a predetermined range (e.g., velocity values in a range between 0 and 1 or any other suitable range). In some examples, different submovement datasets can have different time periods defined by the changes of the movement dataset or the beginning or the end of the movement dataset. In other examples, different submovement datasets can be resampled in time to have the same time period (e.g., to have 40-dimensional vectors or any other suitable vector).

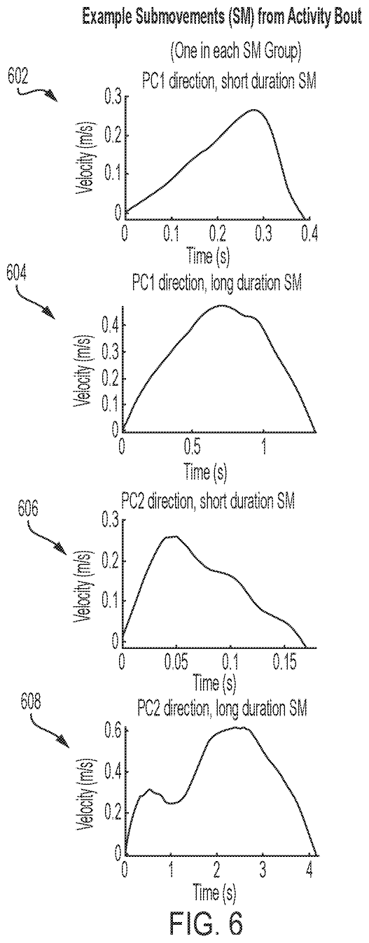

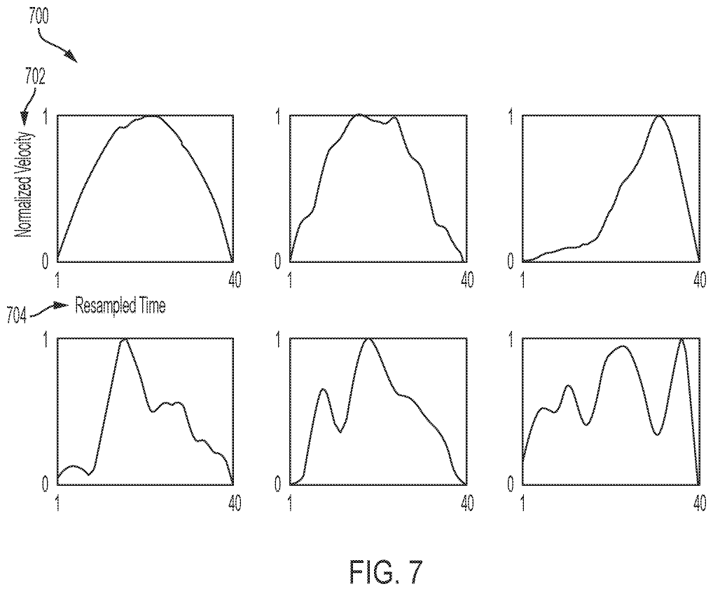

Referring again to FIG. 5, the first principal component dataset 502 of the movement dataset can include negative values 506 (e.g., negative velocity values) and positive values 508 (e.g., positive velocity values). Then, the beginning and/or end of a submovement dataset 510 can be defined by the point where the velocity values of the principal component dataset 502 changes from a positive velocity value to a negative velocity value or from a negative velocity value to a positive velocity value represented by a crossing of the velocity axis in time series. In some examples, the beginning and/or end of some submovement dataset 510 can be defined by the beginning 512 or the end 514 of the first principal component dataset 502. The beginning 512 or the end 514 of the first principal component dataset 502 can have the zero velocity value or can have a different velocity value than the zero value. Thus, the process 200 can generate multiple submovement datasets 510 by dividing the first principal component dataset 502. In further examples, the process 200 can generate multiple other submovement datasets 516 by dividing the second principal component dataset 504 in a similar way to the submovement dataset 510 in the first principal component dataset 502. That is, the process 200 generates multiple submovement datasets 602-608 as shown in FIG. 6. based on the movement dataset (i.e., first and second principal component datasets 502, 504 in FIG. 5). In the examples, the multiple submovement datasets 602-608 can be converted to have positive values. Referring to FIG. 7, the multiple submovement datasets 700 can be normalized to have values 702 in a range between 0 and 1. In further examples, the multiple submovement datasets can be resampled in time 704 to have the same time period to have 40-dimensional vectors.

In further examples, the process 200 can further group the multiple submovement datasets into multiple subsets based on a duration and a direction of the movement of the user in the plurality of submovement datasets. Referring to FIG. 6, the process 200 group the multiple submovement datasets generated at process block 206 into four subsets based on durations (e.g., a long duration and a short duration) and directions (e.g., the primary direction (PC1) and the secondary direction (PC2)) of the submovement datasets. In some examples, the long duration and the short duration of a submovement dataset can be determined by a predetermined threshold. For example, the predetermined threshold can be 0.6 seconds, or any other suitable time period. Thus, in the examples, submovements equal to or below 0.6 seconds are considered to be the short duration while short submovements longer than 0.6 seconds are considered to be the long duration. In further examples, the process 200 can disregard submovements, which are shorter than another threshold or longer than the other threshold. For example, the process 200 can disregard submovements, which are shorter than 0.05 seconds or longer than 5 seconds. In the examples of FIG. 6, submovements 602, 606, which are equal to or shorter than 0.6 seconds are grouped with the short duration while other submovements 604, 608, which are longer than 0.6 seconds are grouped with the long duration. It should be appreciated that the thresholds are not limited to the examples above and can be any suitable threshold durations. In further examples, the directions can include the primary direction and the secondary direction as explained in connection with FIG. 5. Thus, the process 200 can group the multiple submovement datasets into four subsets: 1) PC1 direction with the short duration 602, 2) PC1 direction with the long duration 604, 3) PC2 direction with the short duration 606, and 4) PC2 direction with the long duration 608. It should be appreciated the process 200 can group the multiple submovements into any other suitable number of subsets based on other suitable criteria. The criteria for dividing submovements in groups can be predefined by the user based on properties such as direction, duration, peak velocity, time of day, daytime or nightime, and/or movement type; or can be learned directly from the data, for example using machine learning methods such as clustering.

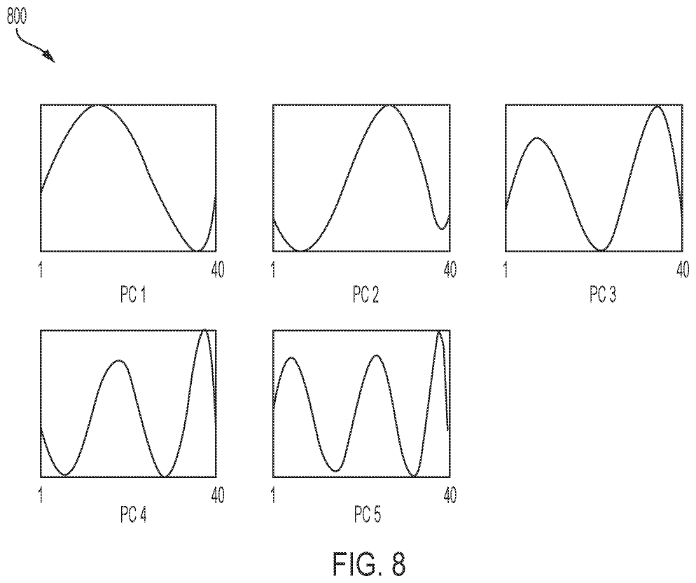

In further examples, principal component analysis (PCA), or another linear or non-linear dimensionality reduction technique, can be used to identify predetermined or machine-learned number (e.g., the top 5) of “basis functions” (PC 1-5) that could be used to optimally reconstruct all normalized submovements. The basis functions can explain the majority of variance in the submovement velocity versus time curve (i.e., submovement shape in FIGS. 6 and 7). In some examples, the principal component (PC) scores for a given submovement can be the linear weights on these five principal component vectors in order to reconstruct the submovement. Thus, the magnitude of PC scores represents how much each principal component contributes to the submovement. Submovements (see FIGS. 6 and 7 for examples) can be partially reconstructed by a linear combination of the five principal component vectors shown. Each panel provides a visualization of an eigenvector, with the element values displayed on the y-axis for each dimension of the 40-dimensional vector. Referring to FIG. 8, PCs 1 and 2 represent low-frequency characteristics of the velocity-time curve and demonstrate a single sine wave cycle with a peak in the first half and second half of the submovement, respectively. PCs 3-5 represent higher frequency characteristics PCs and have increasing cycles in half-cycle increments, with 1.5, 2, and 2.5 cycles, respectively. For clarity, the principal components described here (PCs 1-5), can be used to characterize the shape and curvature of submovement velocity-time curves. Other linear and nonlinear dimensionality reduction techniques (e.g., Isomap, autoencoders) and time series modelling techniques (e.g., linear dynamical systems, wavelet transforms) can be used to perform this step as well. Principal component analysis can be also used for a completely different purpose at an earlier stage (before submovements were identified) to project three dimensional movement in space during an activity bout (FIG. 4) onto a two-dimensional plane and identify the primary (PC 1) and secondary (PC 2) directions of movement (FIG. 5).

Referring again to FIG. 2, at process block 208, the process 200 extracts a movement feature from a first subset of the multiple submovement datasets. In some examples, the first subset of the multiple submovement datasets can be one subset of the four subsets 602-608 in connection with FIG. 6. In other examples, the first subset can be equal to the multiple submovement datasets. In further examples, the movement feature is a representing value of at least one of: distances, peak velocities, peak accelerations, normalized jerks, or durations of the first subset. In even further examples, the representing value can be a mean value, standard deviation value, or other statistic that summarizes the distribution over subsets.

In some examples, a submovement dataset can include a distance (e.g., in meters) traveled. The multiple submovement datasets in a subset can include corresponding distances, and the subset can have a mean value or a standard deviation value for the distances in the subset. Thus, the multiple submovement datasets can have 8 features for submovement distance: 1) a mean value of distances of subset 1 in PC1 direction with the short duration, 2) a mean value of distances of subset 2 in PC1 direction with the long duration, 3) a mean value of distances of subset 3 in PC2 direction with the short duration, 4) a mean value of distances of subset 4 in PC2 direction with the long duration, 5) a standard deviation value of distances of subset 1 in PC1 direction with the short duration, 6) a standard deviation value of distances of subset 2 in PC1 direction with the long duration, 7) a standard deviation value of distances of subset 3 in PC2 direction with the short duration, and 8) a standard deviation value of distances of subset 4 in PC2 direction with the long duration.

In further examples, a submovement dataset can include a peak or maximum velocity (e.g., in m/s). The multiple submovement datasets in a subset can include corresponding peak velocities, and the subset can have a mean value or a standard deviation value for the peak velocities in the subset. Similar to the distance features of the multiple submovement datasets, the multiple submovement datasets can have 8 features for submovement peak velocity: four means values for peak velocities of corresponding subsets (i.e., subset 1, subset 2, subset 3, and subset 4) and four standard deviation values for peak velocities of corresponding subsets (i.e., subset 1, subset 2, subset 3, and subset 4).

In further examples, a submovement dataset can include a peak acceleration (e.g., in m/s²). The multiple submovement datasets in a subset can include corresponding peak accelerations, and the subset can have a mean value or a standard deviation value for the peak accelerations in the subset. Similar to the distance features of the multiple submovement datasets, the multiple submovement datasets can have 8 features for submovement peak acceleration: four means values for peak accelerations of corresponding subsets (i.e., subset 1, subset 2, subset 3, and subset 4) and four standard deviation values for peak accelerations of corresponding subsets (i.e., subset 1, subset 2, subset 3, and subset 4).

In further examples, a submovement dataset can include a normalized jerk, which is dimensionless and scaled based on the submovement duration and submovement peak velocity. The multiple submovement datasets in a subset can include corresponding normalized jerks, and the subset can have a mean value or a standard deviation value for the normalized jerks in the subset. Similar to the distance features of the multiple submovement datasets, the multiple submovement datasets can have 8 features for submovement jerk: four means values for normalized jerks of corresponding subsets (i.e., subset 1, subset 2, subset 3, and subset 4) and four standard deviation values for normalized jerks of corresponding subsets (i.e., subset 1, subset 2, subset 3, and subset 4).

In further examples, a submovement dataset can include a duration (e.g., in seconds). It should be appreciated that the features are not limited to the example features listed above. The process 200 can extract any other suitable movement feature from the multiple submovement datasets. For example, the movement feature can include features that describe the shape and curvature of the submovement velocity-time curve. A submovement can include the principal component 1 (PC1) score. PC1 captures low-frequency characteristics of the SM velocity-time curve (e.g., the SM “shape”). The PC1 “basis function” is a single sinusoidal waveform with the peak present in the first half of the submovement. Mean absolute value, standard deviation, and kurtosis can be computed for long duration submovement datasets in the primary and secondary directions of movement resulting in 3*2=6 total features (applies to SM PC1-5 scores). In further examples, the movement features can include a principal component 2 score. A submovement can include the principal component 2 (PC2) score. Similar to PC1, PC2 captures low-frequency characteristics of the SM velocity-time curve. The PC2 basis function is a single sinusoidal waveform with the peak present in the second half of the submovement. In even further examples, the movement features can include a principal component 3 score, a principal component 4 score, and/or a principal component 5 score. A submovement can include the principal component 3-5 scores. PC3-5 scores can capture higher frequency characteristics of the submovement velocity-time curve. The PC3, PC4, and PC5 basis functions can include 1.5, 2, and 2.5 sinusoidal cycles, respectively. Furthermore, the process 200 can extract features that describe sequences of consecutive submovements, for example the length of the entire sequence, how much time is present between consecutive submovements, and how submovements transition from one subset of submovements to another subset (e.g., using hidden Markov models).

In further examples, the process 200 can extract any other suitable movement feature from the sensor data. For example, a movement feature from the sensor data can include at least one of: an activity intensity (AI) mean, an AI median, an AI mode, an AI entropy, a percentage daytime with low AI, a percentage daytime with moderate AI, a percentage daytime with high AI, a percentage acceleration in single direction, a total power, a bout acceleration, or a bout jerk. The AI mean value is a mean activity index value over all daytime activity over the week-long period. In some examples, periods of inactivity are excluded from calculation of the AI mean, the AI median, the AI mode, and the AI entropy. The AI median is a median activity intensity value over all daytime activity. The AI mode is the mode common value (mode) of activity intensity over all daytime activity. The AI entropy is the entropy of the distribution of daytime activity intensity. The percentage daytime with low AI is the percentage of daytime that is spent performing low intensity movements (0.0045<AI<8.63; a range that includes movement that occurs while sitting quietly and watching television). The percentage daytime with moderate AI (8.63<AI<44.8; a range that includes doing laundry while standing). And the percentage of daytime that is spent performing high intensity movements (44.8<AI<336; a range that includes walking on a treadmill at 2-2.5 miles per hour). Note that other suitable ranges and parsing of activity intensity may be used. To assess how straight movements are in short time intervals (percentage of movement in a single direction), for each one second window (e.g., or any other suitable time window) of movement, principal component analysis can be performed on the triaxial accelerometer data to identify the principal direction of acceleration. This feature is the percentage of accelerometer data variance explained by the first principal component direction, averaged over one second windows. This measure can be computed separately for low AI, moderate AI, and high AI one second windows resulting in three features. The total power is the cumulative power in the 0.1-5 Hz frequency band or any other suitable frequency band. Regarding the bout acceleration, activity bouts are continuous periods of movement activity with durations (e.g., between 4-18 seconds long or any other suitable durations) based on an activity index threshold. Bout acceleration is the maximum acceleration (e.g., in m/s²) during an activity bout. Mean and standard deviation can be computed over a user's activity bouts resulting in two features (applies to bout acceleration and bout jerk). The bout jerk is the mean jerk (derivative of acceleration) (e.g., in m/s³) during an activity bout. Thus, several classes of features can be extracted from the multiple submovement datasets and/or from the sensor data.

In even further examples, the process 200 can extract any other suitable movement feature from other data. For example, the process 200 can extract one or more movement features from eye tracking data, or facial expression/movement data obtained from video data. Computer vision algorithms can be used to extract facial landmark (e.g., forehead, eyebrows, iris, pupil, nose, lips, cheeks, chin, etc.) or body landmark time series data from a video camera present in a mobile device, computer, or a standalone, as an individual uses the device and/or performs their usual daily activities. From this position data over time, activity bouts, submovements, and their characteristics can be obtained and used to quantify motor function, identify early signs of disease, and assess the severity of a disorder.

In further examples, the process 200 can extract one or more movement features from a computer mouse task (Hevelius), which was developed for clinical use in neurological disorders in collaboration with Dr. Krzysztof Gajos. The computer mouse task can include a task for the user to click on targets as soon as the targets appear on the screen. In some scenarios, users can set the minimum size of the target to ensure that the target size is set to a reasonable level of difficulty. The movement feature from the mouse task can include at least one of: a movement time from a first target to a second target, a coefficient value of variation of the movement time, an execution time, a coefficient value of variation of the execution time, an execution time without pauses, a coefficient value of variation of the execution time without pauses, a verification time, a standard deviation value of the verification time, a number of pauses, a duration of longest pause, a max speed, a coefficient value of variation of the max speed, a max acceleration, a coefficient value of variation of the mas acceleration, a normalized jerk, a normalized jerk without pauses, a click duration, a standard deviation value of the click duration, movement direction changes, orthogonal direction changes, task axis crossings, a movement error, a movement offset, a movement variability, a distance from target at end of main submovement, target re-entries, a click slip, a fraction distance covered in main submovement, a fraction of main submovement spent accelerating, a number of submovements, a main submovement, or a noise to force ratio. However, it should be appreciated that the movement feature from the mouse task can be any other suitable feature.

Referring again to FIG. 2, at process block 210, the process 200 analyzes the movement feature from the first subset of the multiple submovement datasets. In one non-limiting example, the analysis at process block 210 may include comparing the first subset of the multiple submovement datasets to a reference to determine a potential clinical disorder of the user or, as will be described, a learning network or neural network may perform the analysis. In some examples, the reference can include a control submovement dataset of a control group without any clinical disorder. The control submovement dataset can have the same movement direction (PC1 or PC2) within the same duration range (long or short duration) as the first subset. The control group can correspond to the user of the multiple submovement datasets such that the ages of the control group are similar to that of the user and the sex of the control group is the same as that of the user. In further examples, the process 200 can determine the potential clinical disorder when the movement feature from the first subset and the movement feature from the control submovement dataset are statistically different with high confidence or are more different than a predetermined threshold. In even further examples, the process 200 can determine the level of severity of the potential clinical disorder based on predetermined thresholds or on a continuous scale. For example, when the movement feature from the first subset for the user and the movement feature from the control submovement dataset are more different than threshold 1 but less different than threshold 2, the process 200 can determine that the potential clinical disorder of the user has level 1 severity of the potential clinical disorder. Similarly, when the movement feature from the first subset for the user and the movement feature from the control submovement dataset are more different than threshold 2 but less different than threshold 3, the process 200 can determine that the potential clinical disorder of the user has level 2 severity of the potential clinical disorder. In some scenarios, ankle submovement (SM) distance, peak velocity, and peak acceleration features are smaller in individuals with ataxia and amyotrophic lateral sclerosis (ALS) compared to controls and become progressively smaller and less variable as disease severity increases. Statistical models may be used to generate a probability of a particular disease or neurological phenotype based on how statistically different a user's submovement characteristics are from the control group and provide recommendations for obtaining further assessment and potentially neurological care.

In further examples, the process 200 uses classification models to generate a prediction about the probability that the user has one or more clinical disorders, based on an aggregation of multiple movement features of the multiple submovement datasets. Then, process 200 can determine the potential clinical disorder of the user based on prediction of a model that was trained to distinguish between individuals with a disease and controls. Thus, the process 200 can determine a potential clinical disorder of the user based on the weighted sum (or other linear or non-linear aggregation) of the multiple features for the multiple submovement datasets of the user. In further examples, process 200 can determine the level of severity of the potential clinical disorder based on the multiple movement features and/or a weighted sum of the multiple movement features. For example, process 200 can impose a larger weight for the submovement distance feature on the subset with the short duration and the secondary direction of the multiple submovement datasets than other subsets. Process 200 can also impose a larger weight for the submovement peak velocity feature on the subsets with the secondary direction of the multiple submovement datasets than other subsets. Process 200 can also impose a larger weight for the submovement peak acceleration feature on the subset with the long duration and the secondary direction of the multiple submovement datasets than other subsets. In further examples, process 200 can impose a larger weight for the AI mean and entropy features than other features.

In further examples, to compare the movement features from the first subset of the plurality of submovement datasets to the reference, the process 200 can obtain a regression model trained with the reference, provide the movement features to the regression model, and generate an output of the regression model to determine the potential clinical disorder of the user. In some examples, the regression model can receive multiple features as input to estimate clinician-rated disease severity (e.g., Brief Ataxia Rating Scale, Unified Parkinson's Disease Rating Scale, Amyotrophic Lateral Sclerosis Functional Rating Scale, etc.). Relatively simple regression models (linear regression with L1 regularization) can be used to promote interpretability or more complex nonlinear regression models can be employed such as Gaussian process regression and random forest regression. In addition to clinician ratings, patient-reported measures of function (e.g., PROM-Ataxia) can be used as the target variable for the motor function estimation model. In further examples, the analysis at process block 210 can be performed by a trained deep learning model or neural network that produces a probability of the potential clinical disorder or the severity level of the potential clinical disorder. In even further examples, the disease severity models can be learned by training a classification model (e.g., logistic regression) to classify the presence of disease progression across two data points collected at different times from the same individual, across a population of individuals with a known degenerative disease. The model weights for this model can then be applied to the submovement features (e.g., without the logistic function applied) to generate a severity score that is independent of clinician or patient-reported measures.

Referring again to FIG. 2, at process block 212, the process 200 generates a report that includes an indication of the potential clinical disorder of the user. In some examples, the indication of the potential clinical disorder of the user is indicative of an estimated severity level of the potential clinical disorder determined based on the output of the regression model. In other examples, the indication of the potential clinical disorder of the user is indicative of existence of the potential clinical disorder determined based on the output of the regression model. In some examples, the process 200 can generate and display the report, via display 114 of the computing device 110 to the user. In other examples, the process 200 can generate and transmit the report, via communications system 118 of the computing device 110 to the communication network 150.

### Examples

The inventor showed, via experiments, that analysis of natural ankle and wrist movements in individuals with spinocerebellar ataxias (SCAs) and multiple system atrophy (MSA) of the cerebellar type (MSA-C) can produce interpretable motor measures that reflect meaningful patient-reported function, have high reliability, and are feasible for use in clinical trials.

38 participants were provided with a study laptop, computer mouse, and web camera to perform the experiments, while four participants used a personal computer that met the experiment criteria. All participants were provided with two wearable sensor devices which collect triaxial accelerometer data at 100 Hz, one for the dominant wrist and one for the dominant ankle.

Each participant's wearable sensor data were manually partitioned into day and night segments based on changes in each participant's daily activity level represented in the accelerometer data. To account for differences in the time of day that sensor recording began across participants, day/night segmentation was started at the onset of the first full night of recording. This produced a maximum of 6 consecutive 24-hour periods of recording. Data analysis focused on daytime segments. Gravity and high frequency noise were removed from the acceleration time-series using a sixth order Butterworth filter with cutoff frequencies of 0.1 and 20 Hz.

Several classes of features were extracted from daytime ankle and wrist sensor data. These included total power in the 0.1-5 Hz frequency range and features based on the distribution of activity intensity computed in 1-second time bins, as per previous work from passive wrist sensor data collection in ataxia-telangiectasia. Features were also extracted from “activity bouts” and from submovements. Activity bouts and submovements were extracted from continuous accelerometer data collected over a 24-hour period. Then, the 85 features extracted were from ankle and wrist sensor data. Single feature analysis was performed on a subset of 26 key features of interest (bolded in Table 2). These included activity intensity (AI) mean (1 feature), AI entropy (1 feature), submovement (SM) distance (8 features), SM velocity (8 features), and SM acceleration (8 features). Mean and standard deviation were computed over a participant's SMs for short duration and long duration SMs in the primary and secondary directions of planar movement resulting in 2*2*2=8 total features

Single feature analysis used a subset of 26 features. However, all 85 ankle sensor features in the experiments were used as input to regression models trained to estimate clinician-rated ataxia severity and patient-reported function. Given the large number of features relative to the number of participants, linear regression models with L1 regularization (i.e., lasso regression) 32 were used to select a small subset of the input variables. Each feature was z-score transformed prior to model training such that feature value ranges and model weights were comparable. BARS total score was used as the target variable for the ataxia severity estimation model as it offered additional granularity with its half-point scores. PROM-Ataxia was used as the target variable for the motor function estimation model. Leave-one-out-cross-validation was used to evaluate performance of the models. Pearson correlation coefficient was used to measure performance, with each model compared with Scale for the Assessment and Rating of Ataxia (SARA) total, SARA gait, BARS total, BARS gait, PROM-Ataxia total, and PROM-Ataxia gait subscore.

Participants were also asked to complete a computer mouse task (Hevelius) twice per week for four weeks (total 8 times). Participants used a mouse to click on targets as soon as they appeared on the screen. During the first study appointment, participants set the minimum size of the target with a study team member to ensure that the target size was set to a reasonable level of difficulty. During a full session of the computer mouse task, participants performed eight rounds of nine targets per round. The task yields 33 features that describe the participant's timing, speed, and accuracy during the task. The task data also enable previously-trained regression models to estimate ataxia and parkinsonism severity and classification models to classify ataxia from control participants. The outputs from these previously-trained models were used in analysis.

The inventor examined properties of motor primitives called “submovements” derived from the continuous wearable sensors in relationship to patient-reported measures of function (PROM-Ataxia) and ataxia rating scales (Scale for the Assessment and Rating of Ataxia and the Brief Ataxia Rating Scale). The test-retest reliability of digital measures and differences between ataxia and control participants were evaluated.

### Results

There were no age differences between ataxia (range: 30-72 years) and control (range: 32-69 years) groups (ρ=0.86). There were 17 female and 17 male participants in the ataxia group and six female and two male participants in the control group. There were no age (ρ=0.15) or SARA total score (ρ=0.42) differences between female and male participants.

The Inventor found strong pairwise correlations between the remote assessment clinical rating scales (BARS, SARA, and Movement Disorder Society-Unified Parkinson Disease Rating Scale (MDS-UPDRS)). BARS was strongly correlated with both SARA (r=0.97) and MDS-UPDRS (r=0.88). BARS, SARA, and MDS-UPDRS demonstrated significant correlations with PROM-Ataxia total score (r=0.75, 0.76, and 0.70, respectively). BARS total demonstrated significant correlations with PROM-Ataxia score subsets of symptoms, motor, arm, and gait (r=0.65, 0.80, 0.80, and 0.81, respectively). SARA total also demonstrated significant correlations with PROM-Ataxia symptoms, motor, arm, and gait subscores (r=0.67, 0.82, 0.80, 0.83, respectively).

Test-retest reliability was high for PROM-Ataxia total score (ICC=0.95), PROM-Ataxia motor subscore (ICC=0.95), and PROM-Ataxia symptom subscore (ICC=0.95), and was moderate for PROM-Ataxia emotion (ICC=0.79) and cognition (ICC=0.71) subscores. For the EQ-5D-5L questionnaire, test-retest reliability was high for the mobility (ICC=0.89), usual activities (ICC=0.82), and anxiety/depression (ICC=0.75) subsections. Test-retest was lower for the pain/discomfort (ICC=0.40) and self-care (ICC=0.62) sections of the survey.

Most ankle submovement (SM) features were significantly correlated with SARA and BARS total scores and gait subscores, PROM-Ataxia total score, and PROM-Ataxia gait subset score (Table 1 below). There were no ankle-sensor based features that were significantly different between female and male participants.

TABLE 1
Properties of ankle sensor features and models

| | | | SM | Direction | Relationship with SARA (BARS) | | | |
|---|---|---|---|---|---|---|---|---|
| | Feature | | Duration | of Motion | Total | | Gait subscore | |
| Sensor | Name | Statistic | Group | Group | r | p-val | r | p-val |
| Ankle | SM | Mean | Long | PC1 | −0.39 (−0.38) | 3.0E−02 | −0.35 (−0.40) | 5.0E−02 |
| (Single | Distance | Mean | Long | PC2 | −0.55 (−0.54) | 2.0E−03 | −0.51 (−0.55) | 3.0E−03 |
| Features) | | Mean | Short | PC1 | −0.62 (−0.64) | 2.0E−04 | −0.69 (−0.68) | 2.0E−05 |
| | | Mean | Short | PC2 | −0.74 (−0.76) | 3.0E−06 | −0.79 (−0.80) | 2.0E−07 |
| | | SD | Long | PC1 | — | n.s. | — | n.s. |
| | | SD | Long | PC2 | −0.43 (−0.44) | 2.0E−02 | −0.41 (−0.46) | 2.0E−02 |
| | | SD | Short | PC1 | −0.69 (−0.72) | 2.0E−05 | −0.75 (−0.76) | 2.0E−06 |
| | | SD | Short | PC2 | −0.79 (−0.80) | 9.0E−07 | −0.83 (−0.85) | 4.0E−08 |
| | SM | Mean | Long | PC1 | −0.63 (−0.63) | 2.0E−04 | −0.61 (−0.66) | 2.0E−04 |
| | Velocity | Mean | Long | PC2 | −0.78 (−0.78) | 8.0E−07 | −0.76 (−0.80) | 7.0E−07 |
| | | Mean | Short | PC1 | −0.58 (−0.61) | 6.0E−04 | −0.66 (−0.66) | 4.0E−05 |
| | | Mean | Short | PC2 | −0.70 (−0.73) | 2.0E−05 | −0.77 (−0.78) | 4.0E−07 |
| | | SD | Long | PC1 | −0.41 (−0.42) | 2.0E−02 | −0.42 (−0.47) | 2.0E−02 |
| | | SD | Long | PC2 | −0.64 (−0.66) | 2.0E−04 | −0.65 (−0.70) | 5.0E−05 |
| | | SD | Short | PC1 | −0.60 (−0.64) | 3.0E−04 | −0.70 (−0.70) | 2.0E−05 |
| | | SD | Short | PC2 | −0.74 (−0.77) | 4.0E−06 | −0.82 (−0.84) | 4.0E−08 |
| | SM | Mean | Long | PC1 | −0.70 (−0.74) | 2.0E−05 | −0.74 (−0.76) | 2.0E−06 |
| | Acceleration | Mean | Long | PC2 | −0.78 (−0.80) | 7.0E−07 | −0.81 (−0.83) | 6.0E−08 |
| | | Mean | Short | PC1 | −0.46 (−0.50) | 8.0E−03 | −0.56 (−0.55) | 7.0E−04 |
| | | Mean | Short | PC2 | −0.58 (−0.63) | 6.0E−04 | −0.68 (−0.68) | 2.0E−05 |
| | | SD | Long | PC1 | −0.47 (−0.52) | 7.0E−03 | −0.56 (−0.58) | 7.0E−04 |
| | | SD | Long | PC2 | −0.62 (−0.65) | 2.0E−04 | −0.71 (−0.72) | 7.0E−06 |
| | | SD | Short | PC1 | — | n.s. | −0.42 (−0.41) | 2.0E−02 |
| | | SD | Short | PC2 | −0.44 (−0.49) | 2.0E−02 | −0.57 (−0.58) | 7.0E−04 |
| | Al | Mean | N/A | N/A | −0.67 (−0.66) | 4.0E−05 | −0.73 (−0.76) | 3.0E−06 |
| | | Entropy | N/A | N/A | −0.72 (−0.72) | 8.0E−06 | −0.78 (−0.80) | 3.0E−07 |

| | | | | | |
|---|---|---|---|---|---|
| Ankle | Ataxia Severity Prediction Model | 0.82 (0.83) | 4.0E−09 | 0.84 (0.88) | 4.0E−10 |
| (Models) | Self-Reported Function Prediction Model | 0.82 (0.82) | 3.0E−09 | 0.82 (0.86) | 4.0E−09 |

| | | | SM | Direction | Relationship with PROM-Ataxia | | | |
|---|---|---|---|---|---|---|---|---|
| | Feature | | Duration | of Motion | Total | | Gait subset | |
| Sensor | Name | Statistic | Group | Group | r | p-val | r | p-val |
| Ankle | SM | Mean | Long | PC1 | −0.60 | 4.0E−04 | −0.54 | 2.0E−03 |
| (Single | Distance | Mean | Long | PC2 | −0.68 | 4.0E−05 | −0.65 | 9.0E−05 |
| Features) | | Mean | Short | PC1 | −0.47 | 7.0E−03 | −0.53 | 3.0E−03 |
| | | Mean | Short | PC2 | −0.62 | 3.0E−04 | −0.66 | 6.0E−05 |
| | | SD | Long | PC1 | −0.58 | 6.0E−04 | −0.50 | 4.0E−03 |
| | | SD | Long | PC2 | −0.66 | 7.0E−05 | −0.59 | 5.0E−04 |
| | | SD | Short | PC1 | −0.60 | 4.0E−04 | −0.64 | 2.0E−04 |
| | | SD | Short | PC2 | −0.74 | 4.0E−06 | −0.75 | 4.0E−06 |
| | SM | Mean | Long | PC1 | −0.77 | 2.0E−06 | −0.72 | 9.0E−06 |
| | Velocity | Mean | Long | PC2 | −0.80 | 4.0E−07 | −0.81 | 3.0E−07 |
| | | Mean | Short | PC1 | −0.39 | 3.0E−02 | −0.45 | 1.0E−02 |
| | | Mean | Short | PC2 | −0.55 | 2.0E−03 | −0.61 | 4.0E−04 |
| | | SD | Long | PC1 | −0.69 | 4.0E−05 | −0.60 | 4.0E−04 |
| | | SD | Long | PC2 | −0.80 | 3.0E−07 | −0.75 | 3.0E−06 |
| | | SD | Short | PC1 | −0.49 | 5.0E−03 | −0.53 | 2.0E−03 |
| | | SD | Short | PC2 | −0.69 | 4.0E−05 | −0.71 | 2.0E−05 |
| | SM | Mean | Long | PC1 | −0.57 | 8.0E−04 | −0.57 | 8.0E−04 |
| | Acceleration | Mean | Long | PC2 | −0.61 | 4.0E−04 | −0.65 | 9.0E−05 |
| | | Mean | Short | PC1 | — | n.s. | — | n.s. |
| | | Mean | Short | PC2 | −0.40 | 3.0E−02 | −0.46 | 8.0E−03 |
| | | SD | Long | PC1 | −0.38 | 3.0E−02 | −0.36 | 4.0E−02 |
| | | SD | Long | PC2 | −0.49 | 5.0E−03 | −0.50 | 4.0E−03 |
| | | SD | Short | PC1 | — | n.s. | — | n.s. |
| | | SD | Short | PC2 | −0.38 | 4.0E−02 | −0.40 | 3.0E−02 |
| | Al | Mean | N/A | N/A | −0.65 | 1.0E−04 | −0.68 | 4.0E−05 |
| | | Entropy | N/A | N/A | −0.65 | 9.0E−05 | −0.70 | 2.0E−05 |

| | | | | | |
|---|---|---|---|---|---|
| Ankle | Ataxia Severity Prediction Model | 0.81 | 9.0E−09 | 0.81 | 7.0E−09 |
| (Models) | Self-Reported Function Prediction Model | 0.83 | 2.0E−09 | 0.83 | 2.0E−09 |

| | | | SM | Direction | Test-retest | | |
|---|---|---|---|---|---|---|---|
| | Feature | | Duration | of Motion | reliability | Disease vs Control | |
| Sensor | Name | Statistic | Group | Group | ICC | p-val | es |
| Ankle | SM | Mean | Long | PC1 | 0.94 | n.s. | — |
| (Single | Distance | Mean | Long | PC2 | 0.95 | 2.0E−02 | 1.2 |
| Features) | | Mean | Short | PC1 | 0.94 | 2.0E−02 | 1.1 |
| | | Mean | Short | PC2 | 0.92 | 5.0E−03 | 1.5 |
| | | SD | Long | PC1 | 0.89 | n.s. | — |
| | | SD | Long | PC2 | 0.87 | n.s. | — |
| | | SD | Short | PC1 | 0.91 | 2.0E−02 | 1.2 |
| | | SD | Short | PC2 | 0.89 | 5.0E−03 | 1.7 |
| | SM | Mean | Long | PC1 | 0.95 | 1.0E−02 | 1.3 |
| | Velocity | Mean | Long | PC2 | 0.95 | 1.0E−02 | 1.7 |
| | | Mean | Short | PC1 | 0.95 | 2.0E−02 | 1.1 |
| | | Mean | Short | PC2 | 0.94 | 5.0E−03 | 1.5 |
| | | SD | Long | PC1 | 0.90 | n.s. | — |
| | | SD | Long | PC2 | 0.90 | 8.0E−03 | 1.4 |
| | | SD | Short | PC1 | 0.92 | 2.0E−02 | 1.2 |
| | | SD | Short | PC2 | 0.89 | 5.0E−03 | 1.7 |
| | SM | Mean | Long | PC1 | 0.96 | 5.0E−03 | 1.6 |
| | Acceleration | Mean | Long | PC2 | 0.94 | 5.0E−03 | 1.8 |
| | | Mean | Short | PC1 | 0.96 | 2.0E−02 | 1.0 |
| | | Mean | Short | PC2 | 0.95 | 9.0E−03 | 1.3 |
| | | SD | Long | PC1 | 0.97 | 2.0E−02 | 1.3 |
| | | SD | Long | PC2 | 0.92 | 4.0E−03 | 1.5 |
| | | SD | Short | PC1 | 0.95 | n.s. | — |
| | | SD | Short | PC2 | 0.90 | 2.0E−02 | 1.2 |
| | Al | Mean | N/A | N/A | 0.88 | 6.0E−03 | 1.5 |
| | | Entropy | N/A | N/A | 0.93 | 8.0E−03 | 1.4 |

| | | | | |
|---|---|---|---|---|
| Ankle | Ataxia Severity Prediction Model | 0.95 | 4.0E−04^{c} | 1.8 |
| (Models) | Self-Reported Function Prediction Model | 0.94 | 6.0E−04^{c} | 1.6 |

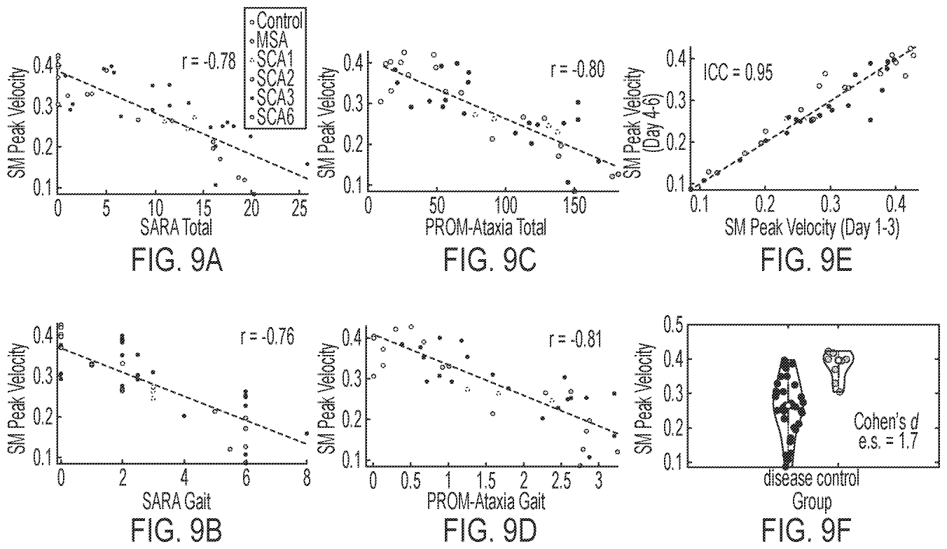

Ankle Submovement Peak Velocity: Referring to FIGS. 9A-9F, properties of a single ankle submovement feature, which is peak velocity of long duration submovement dataset in the secondary direction of the movement, are shown. FIGS. 9A and 9B show the relationship of the feature with the Scale for Assessment and Rating of Ataxia (SARA) total score and gait subscore. FIGS. 9C and 9D show the relationship of the feature with patient-reported measures of function (PROM-Ataxia) total score and gait subscore. As shown in FIGS. 9A-9D, the movement feature (i.e., ankle submovement peak velocity in the secondary direction (PC2 direction)) demonstrated that submovements in the PC2 direction were strongly related to SARA and PROM-Ataxia. The movement feature was informative for both long and short duration subsets or groups of the submovement datasets. Mean peak velocity of the long duration submovment (SM) subset in the PC2 direction was highly negatively correlated with SARA total (r=−0.78 [−0.60:−0.88]), SARA gait subscore (r=−0.76 [−0.57:−0.87]), PROM-Ataxia total (r=−0.80 [−0.63:−0.90]), and PROM-Ataxia gait subscore (r=−0.81 [−0.64:−0.90]). This feature also showed very high test-retest reliability (ICC=0.95) as shown in FIG. 9E and strongly distinguished ataxia and control groups (es=1.7, p<0.01). Variance in peak velocities of both long and short duration SMs in the PC2 direction of movement showed similar properties. FIG. 9F shows disease versus control violin plot. Thus, SM peak velocities became progressively smaller and less variable with decreasing self-reported function and increased ataxia severity, especially for SMs orthogonal to the primary direction of movement.

Ankle Submovement Distance: For ankle SM distance features, short duration submovements in the direction orthogonal to the primary direction of movement (i.e., principal component 2 (PC2) direction) were most strongly related to SARA, BARS, and PROM-Ataxia (see bolded rows in Table 3). Mean distance of this SM group was strongly negatively correlated with SARA total (r=−0.74 [−0.54:−0.86]) and SARA gait subscore (r=−0.79 [−0.61:−0.89]) and was moderately correlated with PROM-Ataxia total (r=−0.62 [−0.35:−0.79]) and PROM-Ataxia gait subscore (r=−0.66 [−0.42:−0.82]). Variance of distances of short duration SMs in the PC2 direction of movement were strongly negatively correlated with SARA total, SARA gait, PROM-ataxia total, and PROM-Ataxia gait (r=−0.79 [−0.61:−0.89], −0.83 [−0.68:−0.91], −0.74 [−0.54:−0.86], and −0.75 [−0.56:−0.87], respectively). These two SM distance features had high test-retest reliability across the first and second half of the week of data collection (ICC=0.89-0.92) and were significantly different between ataxia and control participants (effect size(es)=1.5-1.7, p<0.005). Thus, SM distances were smaller and less variable in individuals with ataxia and became progressively smaller with reduced self-reported function and increased ataxia severity, especially for short duration SMs orthogonal to the primary direction of movement.

Ankle Submovement Peak Acceleration: SM peak acceleration features were informative for longer duration submovements in the PC2 direction, but less so for shorter duration submovements. Mean peak acceleration of this SM group was strongly negatively correlated with SARA total (r=−0.78 [−0.59:−0.88]) and SARA gait subscore (r=−0.81 [−0.65:−0.90]), and moderately correlated with PROM-Ataxia total (r=−0.61 [−0.34:−0.78]) and PROM-Ataxia gait subscore (r=−0.65 [−0.40:−0.81]). This feature showed high test-retest reliability (ICC=0.94) and strongly distinguished ataxia and control groups (es=1.8, p<0.005). All four SM peak acceleration variability features were significantly lower in preataxic individuals (N=4) compared to controls (N=7) with SARA total score <3, although they did not remain significant after correction for multiple comparisons. Out of all 26 individual ankle sensor features, these were the only four that were significantly different between preataxic individuals and controls prior to correction for multiple comparisons.

Ankle Activity Intensity: Activity intensity (AI) mean and entropy were negatively correlated with SARA total (r=−0.67 [−0.43:−0.82] and −0.72 [−0.50:−0.85], respectively), SARA gait subscore (r=−0.73 [−0.52:−0.86], −0.78 [−0.61:−0.89]), PROM total (r=−0.65 [−0.39:−0.81], −0.65 [−0.40:−0.81]), and PROM gait subscore (r=−0.68 [−0.45:−0.83], −0.70 [−0.47:−0.84]). The two AI-based features showed high test-retest reliability (ICC=0.88, 0.93) and were different between ataxia and control participants (es=1.5, 1.4, p<0.01). These findings indicate that ankle movements were progressively less intense with a narrower range of intensity levels as disease severity increased among participants in the study.

Ankle Regression Models: Two separate regression models were trained, one to estimate ataxia severity and one to estimate self-reported function, based on the full set of 85 ankle sensor features. As shown in Table 1 above, the ataxia severity prediction model correlated strongly with SARA total (r=0.82 [0.66-0.91]), SARA gait (r=0.84 [0.71:0.92]), BARS total (r=0.83 [0.68-0.91]), BARS gait (r=0.88 [0.77-0.94]), PROM-Ataxia Total (r=0.81 [0.64:0.90]), and PROM-Ataxia Gait (r=0.81 [0.65:0.90]). The model had very high test-retest reliability (ICC=0.95) and strongly distinguished ataxia and control participants (es=1.8, p<0.001). Both models also were significantly different between preataxic and control participants with SARA total score <3 (es=1.4-1.6, p<0.05). Across all cross-validation folds, the model drew information primarily from only four features: variance in the distance of short duration SMs in the PC2 direction, mean peak velocity of long duration SMs in the PC2 direction, mean jerk during activity bouts, and percent of acceleration data variance explained in a single direction for high activity intensity 1-second windows. The first two selected features were expected based on the single feature analysis. The latter two features, which were not a-priori included in individual feature analysis, indicated that individuals with ataxia had progressively lower mean jerk during activity bouts and a progressively higher percent of triaxial (i.e., three dimensional) acceleration variance explained by a single direction, as disease severity increased. These two features suggest that natural ankle movements become less powerful and less flexible as disease progresses. The four informative features were selected in 100% of cross-validation folds with average model coefficients of −1.49, −1.06, −1.33, and 0.81, respectively. Only three other features were selected in any cross-validation folds; two were selected in 2% of folds and one was selected in 12% of folds. The second regression model that was explicitly trained to estimate self-reported function generated outputs with similar properties (Table 1), however more features were selected across all cross-validation folds (27) with an average of 9.5 features selected per fold.

Continuous Wrist Sensor Data: The majority of wrist submovement distance, velocity, and acceleration features were significantly correlated with SARA, BARS, and PROM-Ataxia. The observed relationships with patient-reported function and ataxia severity were less strong compared to ankle submovements: across all wrist SM features, the strongest correlations with each clinical and patient-reported score were-0.64 [−0.39:−0.81] with SARA total, −0.46 [−0.14:−0.69] with SARA arm, −0.56 [−0.27:−0.75] with BARS arm, −0.66 [−0.42:−0.82] with PROM-Ataxia total, and −0.68 [−0.44:−0.83] with PROM-Ataxia arm. Correlations between wrist sensor features and BARS finger-nose-finger score were stronger and more often statistically significant than correlations with SARA finger-nose-finger score. As with ankle submovements, wrist SM distance, peak velocity, and peak acceleration became progressively smaller and less variable with reduced self-reported function and increased ataxia severity. There were no wrist-sensor based features that were significantly different between female and male participants. Although correlations with clinical scales were lower for the wrist sensor compared with the ankle sensor, many wrist movement features demonstrated very high test-retest reliability. This indicates that reliable information is obtained from the wrist sensor, but it differs substantially from information captured in clinical scales. Longitudinal data is needed to determine if wrist sensor information sensitively captures disease change over time as seen in ataxia-telangiectasia.

Hevelius Computer Mouse Task Data: There were no Hevelius computer mouse task features that were significantly different between female and male participants. Most Hevelius features were significantly correlated with SARA and BARS total scores and arm subscores, PROM-Ataxia total score, and PROM-Ataxia arm subset score. Individuals with ataxia took longer and had more variability in the time to perform each trial of the task. The coefficient of variation (CV) of movement time was strongly positively correlated with SARA total (r=0.85 [0.72-0.92], respectively), SARA arm (r=0.66 [0.41-0.82]), BARS arm (r=0.73 [0.52-0.86]), PROM-Ataxia total (r=0.71 [0.48-0.84]), and PROM-Ataxia arm (r=0.73 [0.52-0.86]). The mean and CV of movement time also showed very high test-retest reliability (ICC 0.99 and 0.94, respectively) and strongly distinguished between ataxia and control participants (es=2.0 and 1.7, p<0.002). The number of pauses and duration of the longest pause were increased in individuals with ataxia and showed similarly strong correlations with ataxia rating scales and self-reported function along with high test-retest reliability. Individuals with ataxia had higher normalized jerk during their mouse movements, and demonstrated reduced accuracy of movements as reflected by larger distances to the target remaining after the main submovement and more target re-entries. The number of movement direction changes was the only feature that was significantly different between preataxic (N=4) and control (N=7) participants with SARA total score <3, however this did not remain significant after correction for multiple comparisons.

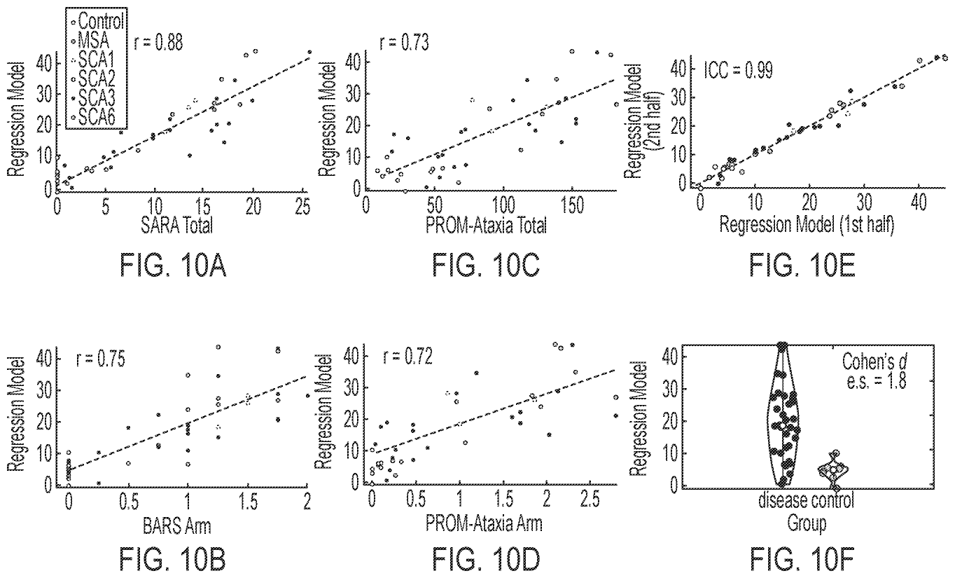

The previously-trained regression model showed particularly strong correlations with SARA total (r=0.88 [0.78-0.94]), BARS arm (r=0.75 [0.55-0.87]), PROM-Ataxia total (r=0.73 [0.52-0.86]), and PROM-Ataxia arm (r=0.72 [0.50-0.85]). This model had an ICC of 0.99 and differentiated ataxia and control participants with an effect size of 1.8 (FIGS. 10A-10F). The previously-trained pairwise comparisons severity estimation models and the classification model were significantly different between preataxic individuals and control participants with SARA total score <3 (es=1.6-2.0, p<0.03). The pairwise comparisons severity estimation models also strongly differentiated all ataxia and control participants (es=2.5-2.7), and had strong relationships with ataxia severity and self-reported function. In FIGS. 10A-10F, properties of a regression model previously trained to estimate parkinsonism severity are shown. This model, which was trained to estimate parkinsonism severity, demonstrated particularly strong relationships with ataxia rating scales, patient reported function, and had high test-retest reliability. FIGS. 10A and 10B shown the relationship of the model with SARA total score and BARS arm subscore. Hevelius task features were consistently more strongly associated with BARS finger-nose-finger score than SARA finger-nose-finger, thus the relationship with BARS arm is shown (see Table 2). FIGS. 10C and 10D shows the relationship of the model with PROM-Ataxia total score and arm subscore. FIG. 10E shows the test-retest reliability of the model while FIG. 10F shows the disease versus control violin plot.

The inventor has shown that digital devices used entirely at home can characterize and quantify self-reported motor function and clinical disorder (e.g., ataxia) with high accuracy and high reliability. In particular, a regression model based on continuous at-home ankle accelerometer data produced a motor measure that strongly correlated with ataxia rating scale total and gait scores (r=0.82-0.88), strongly correlated with self-reported overall and gait function (r=0.81), had high test-retest reliability (ICC=0.95), and distinguished ataxia and control participants, including preataxic individuals. A regression model based on at-home computer mouse task performance produced a motor measure that also strongly correlated with ataxia rating scale total (r=0.86-0.88) and arm scores (r=0.65-0.75), correlated well with self-reported overall and arm function scores (r=0.72-0.73), and had high test-retest reliability (ICC=0.99). These data demonstrate that the assessment technologies provide meaningful and reliable measures of motor function in degenerative ataxias and have population-level sensitivity to disease change. The tools should be evaluated longitudinally in natural history studies to assess individual-level sensitivity to disease progression over time.

Ankle Submovement Characteristics in Ataxia: The ankle sensor used in this study was worn continuously for one week and did not require that participants perform a specific motor task. Interpretation of passively-collected accelerometer data can be challenging without knowledge of the specific behaviors being performed. To address this challenge, data analysis focused on characterizing motor primitives called submovements, extracted automatically from accelerometer data during natural behavior. There is evidence that motor control is achieved by combining elementary submovements to compose voluntary motor behaviors. The concept of movement composition from submovements is of particular relevance in cerebellar ataxias where movements are observed to become segmented or decomposed into constituent parts, potentially due to dyssynchrony of the movement components or as a compensatory strategy to maximize terminal movement accuracy. Thus, submovement-level analysis provides a mechanism to quantify motor impairment-specifically decomposition of movement-without needing to identify specific types of motor behaviors. The inventor found that ankle submovement (SM) distance, peak velocity, and peak acceleration were smaller in ataxia participants compared to controls and became progressively smaller and less variable as self-reported function decreased and ataxia severity increased. Submovements in the plane orthogonal to the primary direction of motion were highly reflective of motor function and ataxia severity; more so than submovements in the primary direction of motion. All four SM acceleration variance measures showed decreased variability in peak acceleration in preataxic individuals compared to controls, although this did not remain significant after correction for multiple comparisons. This pattern of smaller, less powerful, and less flexible submovements in ataxia is consistent with recent descriptions of ankle submovements in adults with ataxia during a gait task, arm submovements in individuals with ataxia during reaching tasks, and wrist submovements in a pediatric genetic ataxia (ataxia-telangiectasia) during natural behavior. These SM changes reflect the hallmark characteristic of the ataxia phenotype that movements become segmented or decomposed into smaller movements. The wrist sensor data presented here also demonstrated progressively smaller SM distance, peak velocity, and peak acceleration, with high test-retest reliability. The SM changes observed were similar to changes seen in healthy older individuals and were in the opposite direction of the changes seen during infant motor development and stroke recovery. Thus, characterization of SMs during natural behavior is also a useful basis for motor assessments in other conditions affecting movement.

Computer Mouse Task Characteristics in Ataxia: The Hevelius computer mouse task was performed twice per week for four weeks (8 times total), requiring the participant to use a mouse to click targets on the screen for 1.3-9.0 (mean=3.8) minutes each time. Individuals with ataxia took longer to perform each trial, had longer and more pauses, and their mouse movements were less smooth and less accurate. The number of movement direction changes were increased in preataxic individuals compared to controls, although this did not remain significant after correction for multiple comparisons. These characteristics are consistent with clinical characterization of the ataxia motor phenotype, prior in-clinic evaluation of computer mouse movements in individuals with ataxia, and evaluation of arm movements in ataxia using other digital technologies. All previously-trained Hevelius regression models showed strong relationships with ataxia rating scales and patient-reported function. The models trained based on pairwise comparisons between individuals with ataxia and parkinsonism were also able to significantly differentiate preataxic and control participants. Interestingly, the regression model previously trained showed the best performance in estimating ataxia severity and participant function. This model strongly weighted mouse movement and click features including task axis crossings, execution time, fraction of the main submovement spent accelerating, number of submovements, max speed, click duration variability, and click slip. The features included in this model have relevance for both parkinsonism and ataxia phenotypes and highlight the utility of creating composite motor measures, which have the potential to be more accurate and reliable than single features.

Reliability of Wearable Sensor and Hevelius Measures: The inventor found that the vast majority of ankle and wrist sensor features had very high test-retest reliability when comparing data from days 1-3 with days 4-6. The two composite regression models trained on ankle data had ICCs of 0.95 and 0.94. The high reliability of SM features and models is driven in part by the aggregation of information over thousands of motor primitives collected from many different behaviors over multiple days. This enables the measures to account for diurnal and daily fluctuations in the disease state. Reliability is expected to be even higher when using data from an entire week.

The Hevelius computer mouse task also showed very high test-retest reliability when comparing the median performance on the task during the first two weeks of the study with the last two weeks. Each session of Hevelius integrates information over 64 trials and median performance over a few sessions (3-4) produced highly reliable motor measurements with an ICC of 0.99 for the UPDRS regression model.

Ecological Validity of Ankle Sensor Measures: Continuous recording of movement using wearable sensors directly captures daily motor behaviors and has the potential to produce measures that closely reflect motor functions that are meaningful to individuals with ataxia. Recent studies in adult ataxias have used a sophisticated 3-sensor system (two ankle sensors and one lumbar sensor) to assess gait and turn characteristics during a several-hour, unsupervised period at home, with ataxia participants instructed to include at least a 30-minute walk (unassisted by walking aids) alongside their usual everyday activities. In these studies, specific gait characteristics including lateral step deviation and spatial step variability were strongly correlated with clinical ataxia severity as measured on SARA gait and posture subscore, with a Spearman p of 0.76. Furthermore, turn characteristics including lateral velocity change and outward acceleration strongly correlated with clinical ataxia severity (ρ=0.79 with SARA total score) and also correlated well with patient-reported balance confidence on the activity-specific balance confidence scale59 (ρ=0.66).

The experiments demonstrated that a single consumer-grade ankle sensor worn continuously for multiple days, without guidelines or restrictions on behavior, can produce measures that closely reflect patient-reported function. The ankle sensor regression models, based on a small number of interpretable submovement characteristics, strongly correlated with patient-reported function, as measured on PROM-Ataxia total and gait subset, with correlation coefficients of 0.81 and 0.83. These correlations with PROM-Ataxia were higher than clinical ataxia rating scale correlations with PROM-Ataxia (SARA: 0.76, BARS: 0.75) and higher than the Hevelius regression model's correlation with PROM-Ataxia (0.73). Correlation of the ankle-sensor based model with SARA total score was also high with a correlation coefficient of 0.82. These observations are consistent with the intuition that information derived from the individual's own selection of behaviors-their typical and natural daily behavior—can accurately, and perhaps most strongly, reflect the individual's own perception of their daily function.

Feasibility and Clinical Applicability: Participants in the study included individuals who were preataxic as well as individuals who used assistive devices such as walkers. Thus the assessment tools were informative and feasible across a wide range of disease stages. While the existing regression models demonstrate strong performance across the spectrum of disease severity, additional models could be trained in the future that are tailored for a specific goal (e.g., estimation of severity in very early disease states).

The motor assessment tools utilized relatively inexpensive and easy-to-use devices. These minimal technological requirements for the at-home assessments may facilitate deployment in clinical studies and increase access.

These data indicate that interpretable, meaningful, and highly reliable motor measures can be obtained from continuous measurement of natural movement, particularly at the ankle location, but also at the wrist location, as individuals perform their daily activities at home. The experiments support the use of these inexpensive and easy-to-use technologies in longitudinal natural history studies in SCAs and MSA-C and show the clinical disorder can be determined based on motor outcome measures in interventional trials.

### Examples

The inventor showed, via an experiment, that movement patterns extracted from continuous wrist accelerometer data, capture motor impairment and disease progression in ataxia-telangiectasia. One week of continuous wrist accelerometer data were collected from 31 individuals with ataxia-telangiectasia and 27 controls aged 2-20 years old. Longitudinal wrist sensor data were collected in 14 ataxia-telangiectasia participants and 13 controls. An example process (e.g., in FIG. 2) was used to extract wrist submovements from the velocity time series. Wrist sensor features were compared with caregiver-reported motor function on the Caregiver Priorities and Child Health Index of Life with Disabilities survey and ataxia severity on the neurologist-performed Brief Ataxia Rating Scale. Submovements became smaller, slower, and less variable in ataxia-telangiectasia compared to controls. High frequency oscillations in submovements were increased and more variable and low frequency oscillations were decreased and less variable in ataxia-telangiectasia. Submovement features correlated strongly with both ataxia severity and caregiver-reported function, demonstrated high reliability, and showed significant progression over a one-year interval. These results show that passive wrist sensor data can produce interpretable and reliable measures that are sensitive to disease change, supporting their potential as ecologically-valid motor biomarkers. The use of a low-cost sensor that is ubiquitous in smartwatches could facilitate participation in neurological care and research regardless of geography and socioeconomic status.

### Results

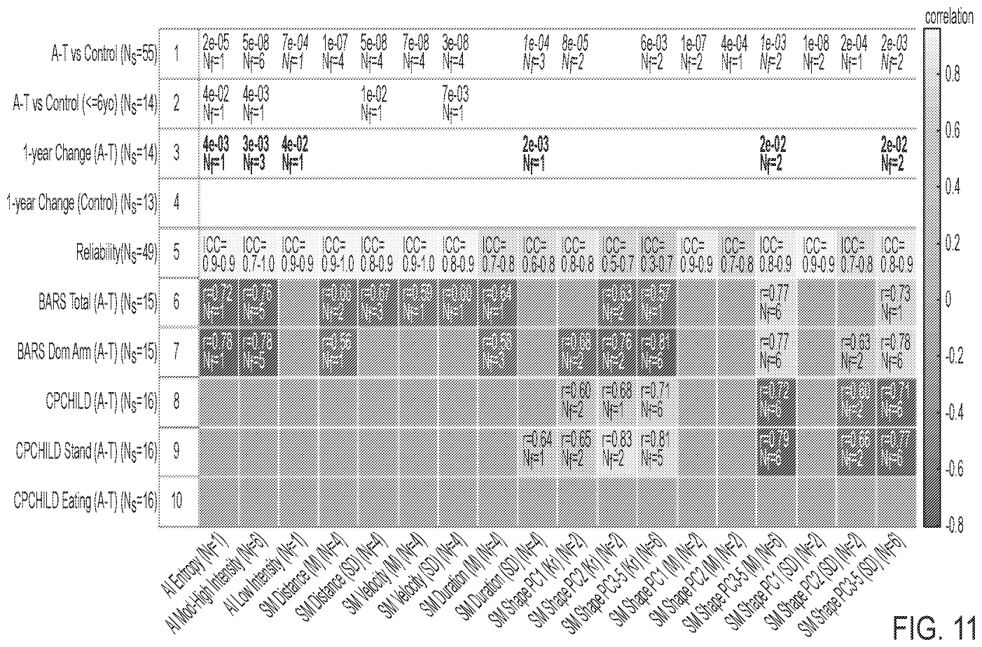

Wrist Sensor Features Differentiate ataxia-telangiectasia and Control Participants: FIG. 11 shows the relationship between each wrist sensor feature subset and key clinical comparisons, including ataxia-telangiectasia (A-T) versus control groups (rows 1-2), change over time in the A-T (row 3) and the control group (row 4), reliability of features for both groups combined (row 5), relationships with the Brief Ataxia Rating Scale (BARS, rows 6-7), and relationships with Caregiver Priorities and Child Health Index of Life with Disabilities (CPCHILD, rows 8-10). Row 4 does not have any text since no features showed statistically significant change in the control group. Rows 1-4 report the number of features (Nf) that are significant within the feature group along with the most significant p-value. Green text indicates when values are higher in the control group compared with the A-T group and red text indicates when the value is higher in A-T compared with controls. Rows 5-10 report the number of significant features along with Intraclass Correlation Coefficients (ICC) or maximum absolute value of the Pearson correlation coefficient (r). The color of each cell also represents the value of the correlation coefficient. In FIG. 11, Nₛ indicates Number of Subjects; Nf indicates Number of Features; AI indicates Activity Intensity; SM indicates Submovement; PC indicates Principal Component; M indicates Mean; SD indicates Standard Deviation; Kr indicates Kurtosis; ICC indicates Intraclass Correlation Coefficient; BARS indicates Brief Ataxia Rating Scale; and CPCHILD indicates Caregiver Priorities and Child Health Index of Life with Disabilities.

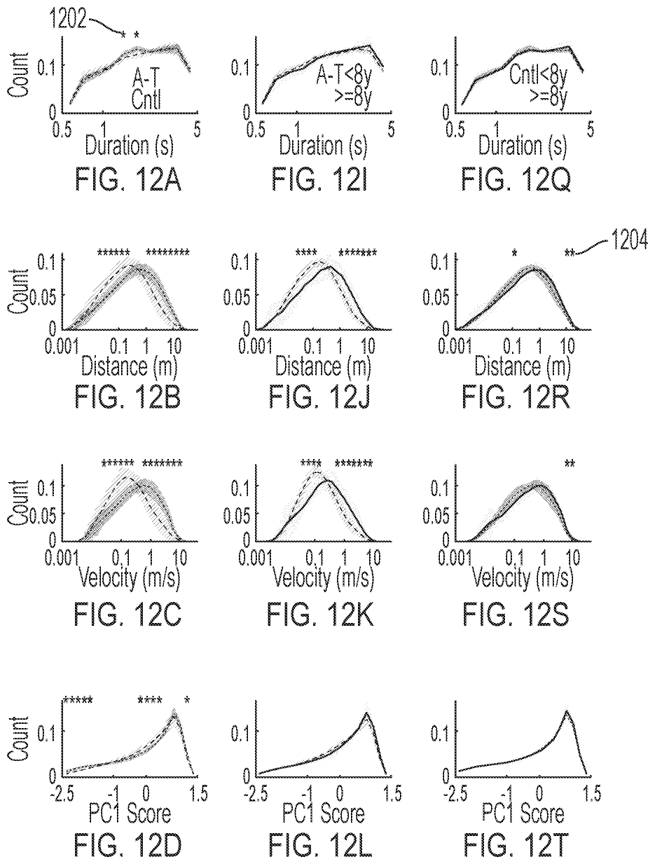

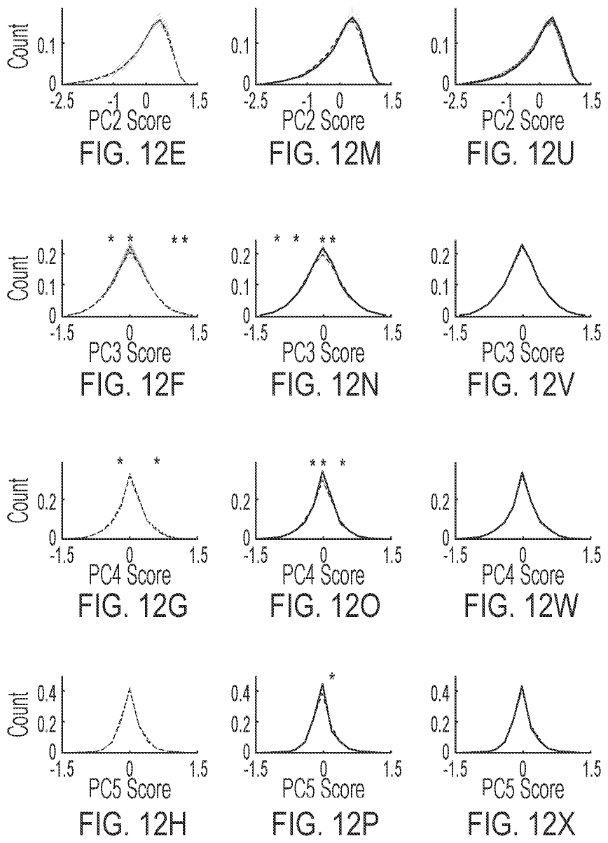

FIGS. 12A-12X show normalized histograms of long duration (0.6-5 second) submovement properties for individuals with A-T versus controls (column 1, 12A-12H), younger versus older individuals with A-T (column 2, 12I-12P), and younger versus older controls (column 3, 12Q-12X). Participants with A-T are shown in red and controls in green. The line is the population group mean and the line width indicates group standard deviation. Duration, distance, and velocity histograms are plotted in log scale. Each histogram bin that is significantly different between groups is marked to indicate the level of significance: p<0.001 (* 1204); p<0.0001 (* 1202).

Sixteen out of the 18 feature groups contained wrist movement features that were significantly different between A-T and control participants (ρ=2×10⁻²-1×10⁻⁸, effect size=0.6-2.3, FIG. 11, row 1). Four out of these 16 subsets had features that remained significantly different when only considering children 6-years-old and younger (ρ=0.042-0.004, effect size=1.2-2.3, FIG. 11, row 2). Individuals with A-T spent more time performing low intensity movements and less time performing high intensity movements, had shorter and less variable submovement distances, and had slower and less variable submovement peak velocities (FIG. 11, row 1; FIGS. 12A-12C, 12I, and 12Q).

A-T submovement velocity versus time profiles (submovement shapes) were also significantly different in A-T and control participants. Both low frequency components (PC 1 and 2 shown in FIG. 8) represented a single sine wave cycle, but the peak of the cycle was in the first half of the submovement for PC 1 and was in the second half for PC 2. The magnitude and variance of PC 1 scores were larger in controls and highly significant in distinguishing A-T and control participants (FIGS. 12A, 12B, 12D, 12I, 12J, 12Q, and 12R). In contrast, A-T submovements had larger and more variable high frequency oscillations (PC 3-5 shown in FIG. 8). The histogram for PC 3 scores was less peaked at zero in A-T compared to controls, consistent with larger high frequency contributions to A-T submovements (FIG. 12F). Thus, submovement velocity-time curves from A-T participants had smaller and less variable low frequency components, particularly at the beginning of the submovement, and larger and more variable high frequency components.

Wrist Sensor Features are Reliable and Capture Disease Progression: Data from 27 participants (14 A-T, 13 controls) were collected at two time points separated by a 1-year interval. Many wrist sensor features demonstrate consistency between the two time points, given that they are derived from several days of continuous movement data. The majority of wrist sensor features showed good to excellent reliability with a median ICC of 0.84 and range of 0.49-0.92 (FIG. 11, row 5). Features were evaluated for their ability to detect disease progression over the 1-year interval. Features from 12 out of the 18 feature groups (21/62 features) demonstrated statistically significant change in the A-T group (FIG. 11, row 3). No features showed significant change in the control group (FIG. 11, row 4). For all 21 features with longitudinal change, the direction of change was congruent with disease progression based on the relationship between the feature and the neurologist-performed ataxia rating scale (BARS). Over the 1-year interval, individuals with A-T had reduced mean activity index (AI), decreased AI entropy, and smaller submovement distances, velocities, and durations. Submovement shapes had larger high frequency oscillations (PC 3-5) and more variability in these high frequency components (FIG. 11, row 3). These longitudinal observations were further supported by cross-sectional age-group differences in the A-T population: older A-T participants (8-years-old and up) had smaller and slower submovements compared with younger A-T participants (FIG. 12J-12K). Additionally, older A-T participants had fewer submovements with no high-frequency components, indicated by a smaller histogram peak at PC 3 and PC 4 scores of zero (FIG. 12N-12O). These age-group changes were not seen for healthy participants (FIG. 12R-12S, 12V-12W), indicating that the changes were largely disease-related rather than age-related changes.

Wrist Sensor Features Correlate with Ataxia Severity and Caregiver-Reported Function: Features from 14 out of 18 feature groups (38/62 features) were significantly correlated with BARS total score (|r|=0.52-0.77, ρ=0.048-0.0007, FIG. 11, row 6) and features from 13/18 groups were significantly correlated with BARS dominant arm score (|r|=0.53-0.81, ρ=0.044-0.0003, FIG. 11, row 7). The wrist movement features that correlated with BARS total and BARS dominant arm were consistent with the features that distinguished A-T from control participants and progressed over time in the A-T group. With increasing ataxia severity, the mean and entropy of activity intensity decreased; submovement distances, velocities, and durations decreased and became less variable; and the mean and variance of high frequency oscillations (PC 3-5) increased (FIG. 11, rows 6 and 7). The mean and variance of PC 2, representing a low frequency velocity peak in the second half of the submovement, also increased with ataxia severity. Although the magnitude and variance of PC 1 scores were larger in controls and highly significant in distinguishing A-T and control participants, they did not correlate with ataxia severity and did not change over time (FIG. 11, rows 3, 6, and 7). On the other hand, kurtosis of the PC 1-3 and 5 distributions, reflecting the amount of probability density outside the central range of the distribution and how heavy-tailed the distributions were, decreased with increasing ataxia severity.

Comparing wrist sensor features with CPCHILD total, standing, and eating scores demonstrated that a subset of wrist sensor features was related to caregiver-reported function (features from 8/14, 8/14, and 3/14 groups, respectively, FIG. 3, rows 8-10). Wrist sensor features had stronger relationships with standing (|r|=0.51-0.83, ρ=0.038-0.00007) and overall motor function (|r|=0.50-0.72, ρ=0.048-0.0015) compared with eating (|r|=0.52-0.59, ρ=0.039-0.033). Whereas activity index and submovement duration and peak velocity features were related to ataxia severity and distinguished A-T from controls, they were not significantly correlated with motor function based on CPCHILD. However, variable and increased high frequency oscillations were significantly correlated with increased motor impairment (FIG. 11, rows 8-9).

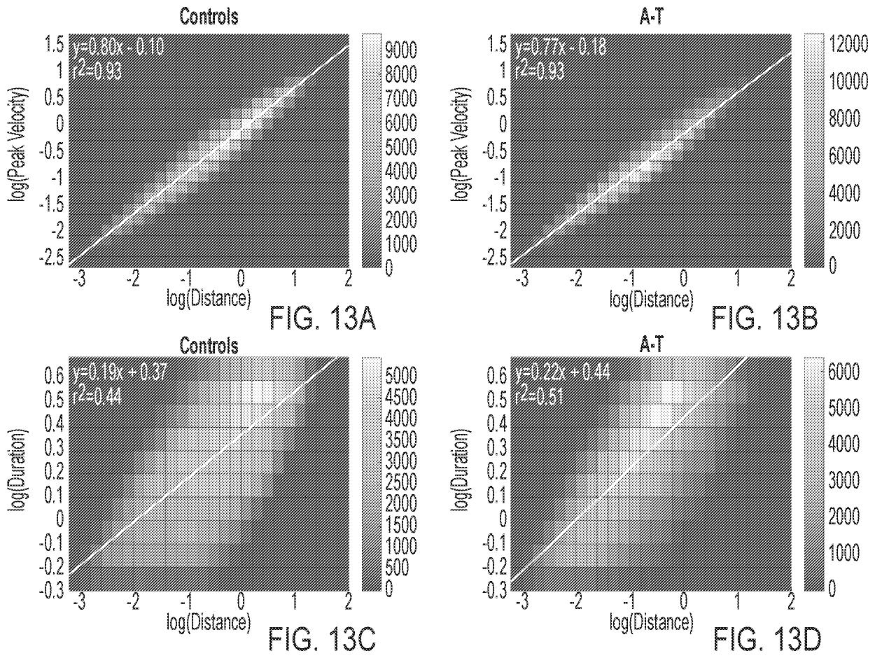

Power Law Relationship between Submovement Velocity and Distance: Prior work has demonstrated a two-thirds power law relationship between submovement velocity and distance during specific motor tasks and a two-thirds power law relationship between curvature and velocity during handwriting and drawing, which may reflect how the motor system plans and optimizes movement. The inventor also observed a strong power law relationship between long duration submovement peak velocity and submovement distance, as indicated by the linear relationship on the log-log 2D histogram (slope: 0.80-0.83, r2: 0.93; FIGS. 13A and 13B), as well as short duration submovements (slope: 0.69-0.73, r2: 0.92-0.94; FIGS. 13A and 13B). The power law relationship was similar for A-T and control groups, however, the center of the 2D distribution was shifted toward smaller and slower submovements for the long duration submovements in A-T, consistent with the 1D distance and velocity histograms in FIGS. 12B and 12C. Referring to FIGS. 13A-13D, two-dimensional, log-log histograms show the relationships between submovement peak velocity and submovement distance (FIGS. 13A and 13B) and submovement duration versus distance (FIGS. 13C and 13D), separately for controls and individuals with A-T for long duration (0.6-5 second) submovements. The linear regression line and equation of each log-log relationship is shown in white. The slope of the line is equivalent to the scaling exponent of the power law relationship between the two variables.

The present invention may be embodied in other specific forms without departing from its spirit or essential characteristics. The described implementations are to be considered in all respects only as illustrative and not restrictive. The scope of the invention is, therefore, indicated by the appended claims rather than by the foregoing description. All changes which come within the meaning and range of equivalency of the claims are to be embraced within their scope.

Discussion: The results demonstrate that real-life, triaxial accelerometer data from a wrist sensor contain reliable and interpretable information about motor impairment in individuals with ataxia-telangiectasia. Features derived from the wrist sensor data distinguished individuals with A-T from controls, had high reliability, detected disease progression over a 1-year interval, and correlated strongly with ataxia rating scales and caregiver-reported function.

The inventor found that both activity intensity (AI) and submovement feature classes carried information relevant to A-T phenotypes. Mean activity intensity and the range of activity intensities were strongly reduced in A-T compared with controls. Additionally, the inventor observed that mean intensity was also significantly reduced in children ≤6 years old and several AI-based features detected disease progression over a 1-year interval. The observed decrease in mean AI and entropy of the AI distribution in A-T participants over time is consistent with the natural history of the disease, which includes slower movements and decreased ability to participate in motor activities over time. While AI-based features correlated with ataxia severity, they did not show statistically significant relationships with caregiver-reported motor function. It is possible that slowing and reducing the intensity of movements assists in the preservation of everyday motor functions, thereby weakening the observed relationship between activity intensity features and caregiver-reported function. It is also possible that CPCHILD doesn't fully capture the motor function changes in A-T and an A-T-specific caregiver-reported outcome tool is needed.

Submovement kinematic features including peak velocity and distance (mean and variance) were strongly reduced in A-T, including in the younger age group. Peak velocity, distance, and duration all decreased with increasing ataxia severity but were not significantly correlated with caregiver-reported motor function. This is consistent with the possibility that reductions in movement speed and distance help maintain motor function, resulting in a weaker observed relationship between the variables. Variability in peak velocity, distance, and duration progressively decreased over a 1-year interval in A-T participants. These findings demonstrate that submovement distance, velocity, and duration decrease in magnitude and become less variable with disease progression in A-T.

Submovement shape features captured both low frequency (PC 1-2) and higher frequency (PC 3-5) oscillations in the velocity-time profile (FIG. 8). The low frequency component with a velocity peak in the first half of the submovement (PC 1) was much weaker and less variable in A-T compared with controls but did not change with increasing motor severity. On the other hand, the low frequency component representing a peak in the second half of the submovement (PC 2), moderately increased and became more variable as ataxia severity and functional impairment increased. Consistent with PC 2, the mean and variance of high frequency (PC 3-5) oscillations increased with worsening ataxia and impaired motor function, and consistently showed progression over a 1-year interval (8/12 features changed in A-T and 0/12 changed in controls, FIG. 11, rows 3-4). The mean and variance of high frequency oscillations consistently showed moderate to strong relationships with BARS total, BARS arm subscore, CPCHILD total, CPCHILD standing, and CPCHILD eating. They also demonstrated high reliability and detected disease change in A-T participants over a 1-year interval. However, they did not distinguish between A-T and control populations as strongly. This shows that high frequency components have different meanings in A-T versus control populations: in controls, high frequency components reflect more flexible and complex movement, whereas in A-T they represent decomposed movements which become increasingly segmented with disease progression.

There is evidence that voluntary movements are composed of motor primitives or submovements that are strung together to form motor behaviors. Submovements have been observed during ballistic reaching movements, slow finger movements, rotary wrist movements, periodic elliptical drawing, and handwriting. Measurements have typically been performed in the laboratory setting using sophisticated equipment such as motion capture systems or robotic arms to record movements. The observations of submovement properties during natural movement are consistent with previously reported properties of submovements during motor tasks. Older individuals appear to compensate for greater noise and lower perceptual efficiency by increasing the number of submovements and decreasing the velocity of submovements during accuracy-constrained movement tasks. During the finger-nose-finger reaching task, individuals with different types of cerebellar ataxia were found to have smaller, shorter, and slower submovements, as well as an increased proportion of submovements with more than one velocity peak. Consistent with these observations, but in the context of improving motor function, healthy infants' reaching trajectories become straighter, and movement units decrease in number and increase in duration, with the dominant unit beginning the movement. In stroke survivors during recovery, the number of submovements decreases and their temporal overlap increases giving rise to smoother trajectories during point-to-point movements. These observations are consistent with the smaller and slower submovements with increased high frequency oscillations observed in A-T with disease progression.

The inventor found a bimodal distribution of submovement durations, motivating separation of submovements into short (0.05-0.6 s) and long (0.6-5 s) duration groups (FIG. 8). The shorter submovements had durations that were largely consistent with those reported in the literature during specific motor tasks. These shorter duration submovements also demonstrated an approximately two-thirds power law relationship between distance and velocity (FIGS. 13A-13D). The longer submovement group included durations that were longer than those reported (mean 0.2-0.44 s, mean 0.2-0.6 s, mean 1.2 s, range 0.05-2.4 s). It is not surprising that there is variation in submovement duration across studies as duration is influenced by motor task parameters and by how submovements are defined and segmented. It is possible that longer duration submovements represent co-articulation or concatenation of movement components and the formation of new movement primitives. If such higher order movement chunks emerge to efficiently represent and control learned movements, one might expect to see these more often during everyday behavior, where well-learned and habitual movements are prevalent. It is also possible that the longer durations the inventor found are in part due to incomplete segmentation of natural behavior submovements. The strong power law relationship between submovement distance and peak velocity (r²=0.93) and the high reliability of submovement shape features over a 1-year period suggest that these long duration submovements are unlikely due to inaccurate segmentation. Furthermore, the strong relationships with ataxia severity and motor function, and consistency with reported changes in older populations, infant development, and stroke recovery, support that longer duration submovements are a meaningful representation of real-life motor behavior.

The wrist movement changes observed in A-T participants indicate that movements become less intense, with a reduced range of intensities, and submovements become smaller, slower, and less variable in their distances and speeds. The primary low frequency component, with a peak in the first half of the submovement velocity profile, is reduced and less variable in A-T. These changes suggest that A-T wrist movements during everyday behavior are decomposed into smaller, less powerful, and less flexible submovements. This reflects a compensatory control mechanism to improve the accuracy and smoothness of movement. These changes could also be in part due to decreased participation in certain types of motor activities. High frequency components contributed more and were more variable in A-T compared with controls. Increased high frequency oscillations were strongly related to ataxia severity and impaired motor function and showed progression over a one-year interval. These larger and more variable high frequency components may reflect flexor-extensor dyssynergy and/or decomposition of movements into smaller primitives as part of a compensatory strategy.

The interpretability, reliability, and sensitivity of movement features extracted from passive wrist sensor data indicates that this technology has potential as an assessment tool and motor outcome measure in A-T clinical trials and clinical care. Importantly, wrist movement characteristics were reflective of overall ataxia severity and motor function, equally or more so than arm-specific ataxia and function subratings. This supports that the motor measurements are ecologically valid and may more closely represent everyday function than measurements from prescribed motor tasks. The consistency of submovement patterns with studies in other populations contributes to the validity of the measures and suggests that they could apply to other neurological populations that affect motor planning and/or execution. As the technology was tested in children as young as 2 years old as well as in individuals who were wheelchair bound, it has potential for application across a wide age range and spectrum of disease severity. Finally, the use of a low-cost, low-burden sensor that is ubiquitous in smartwatches could support participation in neurological care and research for individuals regardless of geography and socioeconomic status.

### Further Examples Having a Variety of Features

The disclosure may be further understood by way of the following examples:
Example 1: A method, apparatus, medical assessment system, and non-transitory computer-readable medium for clinical disorder assessment, comprising: receiving sensor data indicative of movement of the subject; generating a plurality of submovement datasets using the sensor data; extracting a movement feature from a first subset of the plurality of submovement datasets; analyzing the movement feature from the first subset of the plurality of submovement datasets to determine a potential clinical disorder of the user; and generating a report that indicates the potential clinical disorder of the user.
Example 2. The method, apparatus, medical assessment system, and non-transitory computer-readable medium of Example 1, wherein the sensor data includes video or a series of pictures of the user.
Example 3. The method, apparatus, medical assessment system, and non-transitory computer-readable medium of Examples 1 to 2, wherein the clinical disorder includes a neurodegenerative disease.
Example 4. The method, apparatus, medical assessment system, and non-transitory computer-readable medium of any of Examples 1-3, wherein the sensor data includes position data, velocity data or acceleration data.
Example 5. The method, apparatus, medical assessment system, and non-transitory computer-readable medium of any of Examples 1-4, wherein the acceleration data, the position data, or the velocity data is received from one or more wearable sensor devices on at least one of a wrist or an ankle of the user.
Example 6. The method, apparatus, medical assessment system, and non-transitory computer-readable medium of any of Examples 1-5, wherein the acceleration data is derived from video data.
Example 7. The method, apparatus, medical assessment system, and non-transitory computer-readable medium of any of Examples 1-6, further comprising:
reducing dimensions of the sensor data by generating the movement dataset before extracting the movement features.
Example 8. The method, apparatus, medical assessment system, and non-transitory computer-readable medium of any of Examples 1-7, wherein reducing the dimensions of the sensor data comprises: project the sensor data on a two-dimensional plane or a manifold plane.
Example 9. The method, apparatus, medical assessment system, and non-transitory computer-readable medium of any of Examples 1-8, wherein the movement dataset comprises a first principal component dataset in a primary direction, the primary direction having maximum movement variation of the sensor data.
Example 10. The method, apparatus, medical assessment system, and non-transitory computer-readable medium of any of Examples 1-9, wherein the movement dataset further comprises a second principal component dataset in a secondary direction, the secondary direction being orthogonal to the primary direction.
Example 11. The method, apparatus, medical assessment system, and non-transitory computer-readable medium of any of Examples 1-10, wherein generating the plurality of submovement datasets comprises: identifying zero crossing in in the movement dataset; and dividing the movement dataset at each zero crossing to form the plurality of submovement datasets from the movement dataset.
Example 12. The method, apparatus, medical assessment system, and non-transitory computer-readable medium of any of Examples 1-11, wherein a first submovement dataset of the plurality of submovement datasets is a dataset between two abutting zero velocity crossings in the movement dataset.
Example 13. The method, apparatus, medical assessment system, and non-transitory computer-readable medium of any of Examples 1-12, further comprising: grouping the plurality of submovement datasets into a plurality of subsets based on a duration and a direction of the movement of the user in the plurality of submovement datasets, wherein the first subset is among the plurality of subsets.
Example 14. The method, apparatus, medical assessment system, and non-transitory computer-readable medium of any of Examples 1-13, wherein the movement feature is a representing value of at least one of: distances, peak velocities, peak accelerations, or durations of the first subset.
Example 15. The method, apparatus, medical assessment system, and non-transitory computer-readable medium of any of Examples 1, wherein the representing value is a mean value or a standard deviation value.
Example 16. The method, apparatus, medical assessment system, and non-transitory computer-readable medium of any of Examples 1-15, wherein analyzing the movement features from the first subset of the plurality of submovement datasets comprises: obtaining a regression model trained using a reference; providing the movement feature to the regression model; and generating an output of the regression model to determine the potential clinical disorder of the user.
Example 17. The method, apparatus, medical assessment system, and non-transitory computer-readable medium of any of Examples 1-16, wherein the indication of the potential clinical disorder of the user is indicative of an estimated severity level of the potential clinical disorder determined based on the output of the regression model.
Example 18. The method, apparatus, medical assessment system, and non-transitory computer-readable medium of any of Examples 1-17, wherein the indication of the potential clinical disorder of the user is indicative of existence of the potential clinical disorder determined based on the output of the regression model.
Example 19. The method, apparatus, medical assessment system, and non-transitory computer-readable medium of any of Examples 1-18, wherein the potential clinical disorder includes a neurological disorder or a neurodegenerative disease.
Example 20. The method, apparatus, medical assessment system, and non-transitory computer-readable medium of any of Examples 1-19, wherein the potential clinical disorder is ataxia-telangiectasia, spinocerebellar ataxia, multiple system atrophy, or amyotrophic lateral sclerosis.

1. A medical assessment system for clinical disorder assessment, comprising:
an input configured to receive sensor data indicative of movement of a subject;
a memory; and
a processor coupled to the memory;
wherein the processor is configured to:
receive the sensor data indicative of movement of the subject;
generate a plurality of submovement datasets using the sensor data;
extract a movement feature from a first subset of the plurality of submovement datasets;
analyze the movement feature from the first subset of the plurality of submovement datasets to determine a potential clinical disorder of the user; and
generate a report that indicates the potential clinical disorder of the user.

2. The medical assessment system of claim 1, wherein the sensor data includes at least one of:
video or a series of pictures of the user;
position data, velocity data or acceleration data.

3-4. (canceled)

5. The medical assessment system of claim 2, wherein the acceleration data, the position data, or the velocity data is received from one or more wearable sensor devices on at least one of a wrist or an ankle of the user.

6. The medical assessment system of claim 2, wherein the acceleration data is derived from the video data.

7. The medical assessment system of claim 1, wherein the processor is further configured to reduce dimensions of the sensor data by generating the movement dataset before extracting the movement features.

8. The medical assessment system of claim 7, wherein, to reduce the dimensions of the sensor data, the processor is configured to project the sensor data on a two-dimensional plane or a manifold plane.

9. The medical assessment system of claim 7, wherein the movement dataset comprises at least one of:
a first principal component dataset in a primary direction, the primary direction having maximum movement variation of the sensor data; or
a second principal component dataset in a secondary direction, the secondary direction being orthogonal to the primary direction.

10. (canceled)

11. The medical assessment system of claim 7, wherein the processor is configured to generate the plurality of submovement datasets by:
identifying zero crossing in in the movement dataset; and
dividing the movement dataset at each zero crossing to form the plurality of submovement datasets from the movement dataset.

12. The medical assessment system of claim 7, wherein a first submovement dataset of the plurality of submovement datasets is a dataset between two abutting zero velocity crossings in the movement dataset.

13. The medical assessment system of claim 1, wherein the processor is further configured to:
group the plurality of submovement datasets into a plurality of subsets based on a duration and a direction of the movement of the user in the plurality of submovement datasets,
wherein the first subset is among the plurality of subsets.

14. The medical assessment system of claim 1, wherein the movement feature is a representing value of at least one of: distances, peak velocities, peak accelerations, or durations of the first subset.

15. The medical assessment system of claim 14, wherein the representing value is a mean value or a standard deviation value.

16. The medical assessment system of claim 1, wherein to analyze the movement features from the first subset of the plurality of submovement datasets, the processor is configured to:
obtain a regression model trained using a reference;
provide the movement feature to the regression model; and
generate an output of the regression model to determine the potential clinical disorder of the user.

17. The medical assessment system of claim 16, wherein the indication of the potential clinical disorder of the user is indicative of an estimated severity level of the potential clinical disorder determined based on the output of the regression model.

18-19. (canceled)

20. The medical assessment system of claim 1, wherein the potential clinical disorder is ataxia-telangiectasia, spinocerebellar ataxia, multiple system atrophy, or amyotrophic lateral sclerosis.

21. A method for clinical disorder assessment, comprising:
receiving sensor data indicative of movement of the subject;
generating a plurality of submovement datasets using the sensor data;
extracting a movement feature from a first subset of the plurality of submovement datasets;
analyzing the movement feature from the first subset of the plurality of submovement datasets to determine a potential clinical disorder of the user; and
generating a report that indicates the potential clinical disorder of the user.

22. The method of claim 21, wherein the sensor data includes at least one of:
video or a series of pictures of the user; or
position data, velocity data or acceleration data.

23-26. (canceled)

27. The method of claim 21, further comprising:
reducing dimensions of the sensor data by generating the movement dataset before extracting the movement features.

28-32. (canceled)

33. The method of claim 21, further comprising:
grouping the plurality of submovement datasets into a plurality of subsets based on a duration and a direction of the movement of the user in the plurality of submovement datasets,
wherein the first subset is among the plurality of subsets.

34. The method of claim 21, wherein the movement feature is a representing value of at least one of: distances, peak velocities, peak accelerations, or durations of the first subset.

35. (canceled)

36. The method of claim 21, wherein analyzing the movement features from the first subset of the plurality of submovement datasets comprises:
obtaining a regression model trained using a reference;
providing the movement feature to the regression model; and
generating an output of the regression model to determine the potential clinical disorder of the user.

37-38. (canceled)

39. The method of claim 21, wherein the potential clinical disorder includes a neurological disorder or a neurodegenerative disease.

40. (canceled)
